SEQUENCE LISTING (45 sequences)

SEQ ID NO: 1 - protein, 976 aa, Homo sapiens [sequence omitted]

SEQ ID NO: 2 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3 - protein, 976 aa, Artificial Sequence
  Modified CD117 polypeptide
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNVSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 4 - protein, 972 aa, Artificial Sequence
  Modified CD117 polypeptide
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNVSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 5 - protein, 976 aa, Artificial Sequence
  Modified CD117 polypeptide
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFIFAFKGNNKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 6 - protein, 972 aa, Artificial Sequence
  Modified CD117 polypeptide
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFIFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 7 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 8 - protein, 466 aa, Artificial Sequence
  Made in Lab - Heavy chain of an aglycosylated IgG1 humanized antibody
MDWTWRVFCLLAVAPGAHSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 238 aa, Artificial Sequence
  Made in Lab - Light chain of an aglycosylated IgG1 humanized antibody
MVLQTQVFISLLLWISGAYGDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 117 aa, Artificial Sequence
  Made in Lab - variable heavy chain of an aglycosylated IgG1 humanized antibody
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 11 - protein, 111 aa, Artificial Sequence
  Made in Lab - variable light chain of an aglycosylated IgG1 humanized antibody
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 12 - protein, 107 aa, Artificial Sequence
  Made in Lab - variable light chain of humanized monoclonal antibody
  VARIANT (10)..(10): Xaa is an amino acid with an aromatic or aliphatic hydroxyl side chain
  VARIANT (46)..(46): Xaa is an amino acid with an aliphatic or aliphatic hydroxyl side chain
  VARIANT (63)..(63): Xaa is an amino acid with an aliphatic hydroxyl side chain
  VARIANT (80)..(80): Xaa is an amino acid with an aliphatic hydroxyl side chain or is Proline
  VARIANT (85)..(85): Xaa is an amino acid with a charged or acidic side chain
  VARIANT (87)..(87): Xaa is an amino acid with an aromatic side chain
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 13 - protein, 117 aa, Artificial Sequence
  Made in Lab - variable heavy chain of humanized monoclonal antibody
  VARIANT (11)..(11): Xaa is an amino acid with an aliphatic side chain
  VARIANT (20)..(20): Xaa is an amino acid with an aliphatic side chain
  VARIANT (39)..(39): Xaa is an amino acid with a polar or basic side chain
  VARIANT (69)..(69): Xaa is an amino acid with an aliphatic side chain
  VARIANT (71)..(71): Xaa is an amino acid with an aliphatic side chain
  VARIANT (74)..(74): Xaa is an amino acid with an acidic side chain
  VARIANT (83)..(83): Xaa is an amino acid with an acidic or amide derivative side chain
  VARIANT (92)..(92): Xaa is an amino acid with an aliphatic hydroxyl side chain
QVQLVQSGAEXKKPGASVKXSCKASGYTFTDYYINAVVXQAPGKGLEWIARIYPGSGNTYYNEKFKGRXTXTAXKSTSTAYMXLSSLRSEDXAVYFCARGVYYFDYWGQGTTVTVSS

SEQ ID NO: 14 - protein, 116 aa, Artificial Sequence
  Made in Lab - VH domain of humanized antibody
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSS

SEQ ID NO: 15 - protein, 116 aa, Artificial Sequence
  Made in Lab - VH domain of humanized antibody
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSA

SEQ ID NO: 16 - protein, 108 aa, Artificial Sequence
  Made in Lab - VL domain of humanized antibody
DIVMTQSQKFMSTSVGDRVSVTCKASQNVRTNVAWYQQKPGQSPKALIYSASYRYSGVPDRFTGSGSGTDFTLTISNVQSEDLADYFCQQYNSYPRTFGGGTKLEIKR

SEQ ID NO: 17 - protein, 31 aa, Homo sapiens
EKAEATNTGKYTCTNKHGLSNSIYVFVRDPA

SEQ ID NO: 18 - protein, 21 aa, Homo sapiens
RCPLTDPEVTNYSLKGCQGKP

SEQ ID NO: 19 - protein, 123 aa, Artificial Sequence
  Made in Lab - heavy chain variable region of engineered antibody
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 20 - protein, 107 aa, Artificial Sequence
  Made in Lab - light chain variable region of engineered antibody
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 21 - protein, 117 aa, Artificial Sequence
  Made in Lab - heavy chain variable region of engineered antibody
QVQLVQSGAEVKKPGASVKVSCKASGYTFTSYMNHWVRQAPGQGLEWMGVIYSGNGDTSYAQKFKGRVTITADKSTSTAYMELSSLRSEDTAVYYCARERDTRFGNWGQGTLVTVSS

SEQ ID NO: 22 - protein, 117 aa, Artificial Sequence
  Made in Lab - heavy chain variable region of engineered antibody
QVQLVQSGAEVKKPGASVKVSCKASGYTFTSYMNHWVRQAPGQGLEWMGVIYSGNGDTSYNQKFQGRVTITADKSTSTAYMELSSLRSEDTAVYYCARERDTRFGNWGQGTLVTVSS

SEQ ID NO: 23 - protein, 117 aa, Artificial Sequence
  Made in Lab - heavy chain variable region of engineered antibody
QVQLVQSGAEVKKPGASVKVSCKASGYTFTSYMNHWVRQAPGQGLEWMGVIYSGNGDTSYAQKFQGRVTITADKSTSTAYMELSSLRSEDTAVYYCARERDTRFGNWGQGTLVTVSS

SEQ ID NO: 24 - protein, 111 aa, Artificial Sequence
  Made in Lab - light chain variable region of engineered antibody
DIVMTQSPLSLPVTPGEPASISCRASESVDIYGQSFMHWYQQKPGQPPKLLIYLASNLESGVPDRFSGSGSGTDFTLKISRVEAEDVGVYYCQQNNEDPYTFGGGTKVEIK

SEQ ID NO: 25 - protein, 4 aa, Homo sapiens
GNNK

SEQ ID NO: 26 - protein, 4 aa, Artificial Sequence
  Made in lab - VH CDR1 of aglycosylated IgG1 humanized antibody
YNMH

SEQ ID NO: 27 - protein, 16 aa, Artificial Sequence
  Made in lab - VH CDR2 of aglycosylated IgG1 humanized antibody
IYSGNGDTSYNQKFKG

SEQ ID NO: 28 - protein, 8 aa, Artificial Sequence
  Made in lab - VH CDR3 of aglycosylated IgG1 humanized antibody
ERDTRFGN

SEQ ID NO: 29 - protein, 15 aa, Artificial Sequence
  Made in lab - VL CDR1 of aglycosylated IgG1 humanized antibody
RASESVDIYGNSFMH

SEQ ID NO: 30 - protein, 7 aa, Artificial Sequence
  Made in lab - VL CDR2 of aglycosylated IgG1 humanized antibody
LASNLES

SEQ ID NO: 31 - protein, 9 aa, Artificial Sequence
  Made in lab - VL CDR3 of aglycosylated IgG1 humanized antibody
QQNNEDPYT

SEQ ID NO: 32 - protein, 5 aa, Artificial Sequence
  Made in lab - VH CDR1 of engineered antibody
NYWIG

SEQ ID NO: 33 - protein, 17 aa, Artificial Sequence
  Made in lab - VH CDR2 of  engineered antibody
IINPRDSDTRYRPSFQG

SEQ ID NO: 34 - protein, 14 aa, Artificial Sequence
  Made in lab - VH CDR3 of  engineered antibody
HGRGYEGYEGAFDI

SEQ ID NO: 35 - protein, 11 aa, Artificial Sequence
  Made in lab - VL CDR1 of  engineered antibody
RSSQGIRSDLG

SEQ ID NO: 36 - protein, 7 aa, Artificial Sequence
  Made in lab - VL CDR2 of  engineered antibody
DASNLET

SEQ ID NO: 37 - protein, 9 aa, Artificial Sequence
  Made in lab - VL CDR3 of engineered antibody
QQANGFPLT

SEQ ID NO: 38 - protein, 5 aa, Artificial Sequence
  Made in lab - VH CDR1 of engineered antibody
SYNMH

SEQ ID NO: 39 - protein, 17 aa, Artificial Sequence
  Made in lab - VH CDR2 of  engineered antibody
  VARIANT (12)..(12): Xaa is Ala or Asn
  VARIANT (16)..(16): Xaa is Lys or Gln
VIYSGNGDTSYXQKFXG

SEQ ID NO: 40 - protein, 8 aa, Artificial Sequence
  Made in lab - VH CDR3 of  engineered antibody
ERDTRFGN

SEQ ID NO: 41 - protein, 15 aa, Artificial Sequence
  Made in lab - VL CDR1 of  engineered antibody
  VARIANT (4)..(4): Xaa is Asp or Glu
  VARIANT (11)..(11): Xaa is Asn or Gln
RASXSVDIYGXSFMH

SEQ ID NO: 42 - protein, 7 aa, Artificial Sequence
  Made in lab - VL CDR2 of  engineered antibody
LASNLES

SEQ ID NO: 43 - protein, 9 aa, Artificial Sequence
  Made in lab - VL CDR3 of engineered antibody
QQNNEDPYT

SEQ ID NO: 44 - protein, 17 aa, Artificial Sequence
  Made in lab - VH CDR2 of  engineered antibody
VIYSGNGDTSYAQKFKG

SEQ ID NO: 45 - protein, 15 aa, Artificial Sequence
  Made in lab - VL CDR1 of  engineered antibody
RASESVDIYGQSFMH

(19) United States
(12) Patent Application Publication
(10) Pub. No.: US 2024/0115615 A1
(43) Pub. Date: Apr. 11, 2024

(54) MODIFIED STEM CELL COMPOSITIONS AND METHODS FOR USE

(71) Applicant: Jasper Therapeutics, Inc., Redwood City, CA (US)

(72) Inventors: Wendy PANG, Palo Alto, CA (US); Judith SHIZURU, Palo Alto, CA (US); Robert SIKORSKI, Woodside, CA (US); Rajiv TIWARI, Alhambra, CA (US); Hye-Sook KWON, San Mateo, CA (US)

(21) Appl. No.: 18/276,325

(22) PCT Filed: Feb. 9, 2022
(86) PCT No.: PCT/US2022/015861

§ 371 Date: Aug. 8, 2023

Related U.S. Application Data
(60) Provisional application No. 63/147,627, filed on Feb. 9, 2021.
(60) Provisional application No. 63/257,012, filed on Oct. 18, 2021.

(51) Int. Cl. A61K 35/28; C07K 14/705; C12N 5/0789

(52) U.S. Cl. CPC A61K 35/28; C07K 14/70503; C12N 5/0647; C12N 2510/00

(57) ABSTRACT

Modified stem cells and methods of use for stem cell transplant are provided.

### RELATED APPLICATIONS

This application claims priority to U.S. Provisional Patent Application Ser. No. 63/147,627, filed Feb. 9, 2021, and U.S. Provisional Patent Application Ser. No. 63/257,012, filed Oct. 18, 2021, which are incorporated herein by reference in their entireties.

### SEQUENCE LISTING

This application is being filed electronically via EFS-Web and includes an electronically submitted sequence listing in .txt format. The .txt file contains a sequence listing entitled JATH_001_01WO_ST25.txt created on Feb. 8, 2022 and having a size of 82 kilobytes. The sequence listing contained in this .txt file is part of the specification and is incorporated herein by reference in its entirety.

### FIELD OF THE INVENTION

The present disclosure relates to modified hematopoietic stem cells, e.g., comprising a constitutively active modified CD117, and their use for hematopoietic stem cell transplantation.

### BACKGROUND

Hematopoietic cell transplantation (HCT) generally involves the intravenous infusion of autologous or allogeneic donor hematopoietic stem cells (HSCs) or hematopoietic stem and progenitor cells (HSPCs) obtained from bone marrow, peripheral blood, or umbilical cord blood into a subject whose bone marrow or immune system is damaged or defective. HCT may be performed as part of therapy to treat a number of disorders, including cancers, such as leukemias, and congenital immunodeficiency disorders.

HCT is usually accompanied by a preparative or conditioning regimen to clear bone-marrow niches of endogenous HSCs, in order for donor HSCs to engraft. Current conditioning regimens may include treatment with DNA damaging radiation and/or chemotherapy, which can have toxic effects that limit the use of HCT. More recently, a non-genotoxic approach of targeting and depleting HSC has been developed, which uses an antibody that binds human CD117 (c-Kit), a receptor tyrosine kinase expressed on the surface of HSC and progenitor cells (HSPC). Treatment with the anti-c-Kit antibody has been shown to suppress human hematopoiesis in vitro, deplete human HSC in mice xenografted with human cells, and safely deplete HSC of non-human primates (Agarwal, R. et al., Blood (2019) 134 (Supplement_1): 800.

Nonetheless, there remains a need in the art for improved compositions and methods for HCT, including conditioning methods with reduced toxicity and increased engraftment of transplanted HSCs and/or HSPCs. The present disclosure addresses this need.

### BRIEF SUMMARY OF THE INVENTION

The present disclosure provides inter alia novel modified CD117 polypeptides and related compositions and methods of use thereof in hematopoietic stem cell transplant. In particular embodiments, the modified CD117 polypeptides are capable of signaling in HSCs and/or HSPCs in the absence of stem cell factor (SCF) binding. In certain embodiments, the modified CD117 polypeptides provide for constitutive signaling and/or CD117-mediated kinase activity when expressed or present in cells, e.g., HSCs and/or HSPCs. Accordingly, in particular embodiments, when expressed or present in HSCs and/or HSPCs, the modified CD117 polypeptides allow CD117 signaling when bound by antibodies that block SCF binding to CD117.

In one aspect, the disclosure provides a modified CD117 polypeptide comprising one or more amino acid modifications as compared to a wild type CD117 polypeptide, e.g., one or more amino acid substitutions, insertions, or deletions. In certain embodiments, the modified CD117 polypeptide comprises one or more amino acid substitutions, e.g., at one or more of the following amino acids present in wild type human CD117: N505 or D816, such as, e.g., a D816V substitution and/or a N505I substitution. In particular embodiments, the one or more amino acid modifications is located within surface exposed amino acid residues or regions of the extracellular domain, the membrane spanning region, or the intracellular domain of the wild type CD117 polypeptide, e.g., the juxtamembrane region or a kinase domain. In particular embodiments, the modified CD117 polypeptide has at least 90%, at least 95%, at least 98%, or at least 99% sequence homology to the wild type CD117 polypeptide, or a functional fragment thereof. In certain embodiments, the wild type CD117 polypeptide is a wild type human CD117 polypeptide, optionally having one of the following amino acid sequences:

(SEQ ID NO: 1)
MRGARGAWDFLCVLLLLLRVQTGSSQPSVSPGEPSPPSIHPGKSDLIVR
VGDEIRLLCTDPGFVKWTFEILDETNENKQNEWITEKAEATNTGKYTCT
NKHGLSNSIYVFVRDPAKLFLVDRSLYGKEDNDTLVRCPLTDPEVTNYS
LKGCQGKPLPKDLRFIPDPKAGIMIKSVKRAYHRLCLHCSVDQEGKSVL
SEKFILKVRPAFKAVPVVSVSKASYLLREGEEFTVTCTIKDVSSSVYST
WKRENSQTKLQEKYNSWHHGDFNYERQATLTISSARVNDSGVFMCYANN
TFGSANVTTTLEVVDKGFINIFPMINTTVFVNDGENVDLIVEYEAFPKP
EHQQWIYMNRTFTDKWEDYPKSENESNIRYVSELHLTRLKGTEGGTYTF
LVSNSDVNAAIAFNVYVNTKPEILTYDRLVNGMLQCVAAGFPEPTIDWY
FCPGTEQRCSASVLPVDVQTLNSSGPPFGKLVVQSSIDSSAFKHNGTVE
CKAYNDVGKTSAYFNFAFKGNNKEQIHPHTLFTPLLIGFVIVAGMMCII
VMILTYKYLQKPMYEVQWKVVEEINGNNYVYIDPTQLPYDHKWEFPRNR
LSFGKTLGAGAFGKVVEATAYGLIKSDAAMTVAVKMLKPSAHLTEREAL
MSELKVLSYLGNHMNIVNLLGACTIGGPTLVITEYCCYGDLLNFLRRKR
DSFICSKQEDHAEAALYKNLLHSKESSCSDSTNEYMDMKPGVSYVVPTK
ADKRRSVRIGSYIERDVTPAIMEDDELALDLEDLLSFSYQVAKGMAFLA
SKNCIHRDLAARNILLTHGRITKICDFGLARDIKNDSNYVVKGNARLPV
KWMAPESIFNCVYTFESDVWSYGIFLWELFSLGSSPYPGMPVDSKFYKM
IKEGFRMLSPEHAPAEMYDIMKTCWDADPLKRPTFKQIVQLIEKQISES
TNHIYSNLANCSPNRQKPVVDHSVRINSVGSTASSSQPLLVHDDV;
or
(SEQ ID NO: 2)
MRGARGAWDFLCVLLLLLRVQTGSSQPSVSPGEPSPPSIHPGKSDLIVR
VGDEIRLLCTDPGFVKWTFEILDETNENKQNEWITEKAEATNTGKYTCT
NKHGLSNSIYVFVRDPAKLFLVDRSLYGKEDNDTLVRCPLTDPEVTNYS
LKGCQGKPLPKDLRFIPDPKAGIMIKSVKRAYHRLCLHCSVDQEGKSVL
SEKFILKVRPAFKAVPVVSVSKASYLLREGEEFTVTCTIKDVSSSVYST
WKRENSQTKLQEKYNSWHHGDFNYERQATLTISSARVNDSGVFMCYANN
TFGSANVTTTLEVVDKGFINIFPMINTTVFVNDGENVDLIVEYEAFPKP
EHQQWIYMNRTFTDKWEDYPKSENESNIRYVSELHLTRLKGTEGGTYTF
LVSNSDVNAAIAFNVYVNTKPEILTYDRLVNGMLQCVAAGFPEPTIDWY
FCPGTEQRCSASVLPVDVQTLNSSGPPFGKLVVQSSIDSSAFKHNGTVE
CKAYNDVGKTSAYFNFAFKEQIHPHTLFTPLLIGFVIVAGMMCIIVMIL
TYKYLQKPMYEVQWKVVEEINGNNYVYIDPTQLPYDHKWEFPRNRLSFG
KTLGAGAFGKVVEATAYGLIKSDAAMTVAVKMLKPSAHLTEREALMSEL
KVLSYLGNHMNIVNLLGACTIGGPTLVITEYCCYGDLLNFLRRKRDSFI
CSKQEDHAEAALYKNLLHSKESSCSDSTNEYMDMKPGVSYVVPTKADKR
RSVRIGSYIERDVTPAIMEDDELALDLEDLLSFSYQVAKGMAFLASKNC
IHRDLAARNILLTHGRITKICDFGLARDIKNDSNYVVKGNARLPVKWMA
PESIFNCVYTFESDVWSYGIFLWELFSLGSSPYPGMPVDSKFYKMIKEG
FRMLSPEHAPAEMYDIMKTCWDADPLKRPTFKQIVQLIEKQISESTNHI
YSNLANCSPNRQKPVVDHSVRINSVGSTASSSQPLLVHDDV.

In particular embodiments, the modified CD117 polypeptide substantially retains kinase activity as compared to the wild type CD117 polypeptide, and in some embodiments, the modified CD117 polypeptide substantially has increased kinase activity as compared to the wild type CD117 polypeptide. In particular embodiments, the modified CD117 polypeptide substantially retains or has increased kinase activity, optionally constitutive kinase activity, in the absence of SCF binding, as compared to the kinase activity of wild type CD117 polypeptide in the presence of SCF binding. In particular embodiments, the modified CD117 polypeptide substantially retains kinase activity, optionally in response to SCF binding, as compared to the wild type CD117 polypeptide. In particular embodiments, the modified CD117 polypeptide has increased kinase activity, optionally in response to SCF binding, as compared to the wild type CD117 polypeptide. In particular embodiments, the modified CD117 has constitutive kinase activity, optionally in the absence of SCF binding, of at least 30%, at least 40%, at least 50%, at least 60%, at least 70%, at least 80%, at least 90%, or at least 95% of kinase activity in response to SCF binding in cells, e.g., HSCs and/or HSPCs, expressing the wild type CD117 polypeptide.

In particular embodiments, the one or more amino acid modifications do not substantially inhibit or reduce c-Kit signaling or cell proliferation, optionally but not necessarily in response to SCF binding, by the modified CD117 polypeptide expressed in cells, e.g., HSCs and/or HSPCs, as compared to the wild type CD117 polypeptide.

In particular embodiments, the one or more amino acid modifications do not substantially inhibit or reduce binding of an anti-c-Kit antibody to the modified CD117 polypeptide expressed in cells, e.g., HSCs and/or HSPCs, as compared to the wild type CD117 polypeptide. In certain embodiments, the anti-c-Kit antibody disrupts or blocks binding of SCF to wild type CD117 and/or the modified CD117 polypeptide. In particular embodiments, the anti-c-Kit antibody comprises the six CDRs present in any one of JSP191, AB85, CDX-0159, or FSI-174. In particular embodiments, the anti-c-Kit antibody in any one of JSP191, AB85, CDX-0159, or FSI-174.

In particular embodiments, the one or more amino acid modifications do not substantially inhibit or reduce binding of stem cell factor (SCF) to the modified CD117 polypeptide expressed in cells as compared to the wild type CD117 polypeptide.

In a related embodiment, the disclosure provides a nucleic acid encoding the modified CD117 polypeptide. In particular embodiments, the nucleic acid comprises RNA, DNA, or a combination thereof. In one embodiment, the nucleic acid comprises a modified mRNA. In particular embodiments, the nucleic acid is associated with one or more lipids, optionally wherein the nucleic acid is present within a lipid nucleic acid particle, a lipid nanoparticle, or a liposome.

In a further related embodiment, the disclosure provides a vector comprising the nucleic acid encoding the modified CD117 polypeptide. In certain embodiments, the vector is an expression vector, e.g., an AAV vector or a lentiviral vector. In particular embodiments, the vector is capable of transducing hematopoietic stem cells. In related embodiments, the disclosure provides a host cell comprising a vector comprising the nucleic acid encoding the modified CD117 polypeptide. In particular embodiments, the host cell is a bacterial or mammalian cell.

In another related embodiment, the disclosure provides a modified cell comprising the modified CD117 polypeptide and/or the nucleic acid encoding the modified CD117 polypeptide. In particular embodiments, the cell expresses both the modified CD117 polypeptide and wild type CD117 polypeptide. In certain embodiments, the cell was transduced with the vector. In certain embodiments, the cell is a stem cell. In certain embodiments, the cell is a hematopoietic stem and progenitor cell (HSPC) or a hematopoietic stem cell (HSC). In particular embodiments, the cell is CD34+, and in some embodiments, the cell is CD34+/CD90+, CD34+/CD38−, CD34+/CD38−/CD90+, or CD34+/CD133+. In some embodiments, the cell is a human cell. In some embodiments, the cell was obtained from a mammalian donor. In certain embodiments, the mammalian donor is a subject in need of a hematopoietic stem cell transplant (autologous donor), wherein in other embodiments, the mammalian donor is not the subject in need of the hematopoietic stem cell transplant (allogeneic donor). In certain embodiments, the cell expresses the modified CD117 polypeptide, optionally wherein the modified cell expresses the modified CD117 polypeptide transiently. In particular embodiments, the modified CD117 polypeptide is expressed on the cell surface or in the cell membrane, and in certain embodiments, the cell is capable of proliferating in the presence of an anti-CD117 antibody and/or in the absence of SCF. In certain embodiments, the anti-CD117 antibody is capable of inhibiting proliferation and/or survival of a cell expressing only the wild-type CD117 but does not substantially inhibit proliferation and/or survival of a cell expressing the modified CD117 polypeptide. In some embodiments, the anti-CD117 antibody induces apoptosis or death of a cell expressing only the wild-type CD117 but does not substantially induce apoptosis or death of a cell expressing the modified CD117 polypeptide. In certain embodiments, contact with or the presence of the anti-CD117 antibody results in less than 50%, less than 40%, less than 30%, less than 20%, or less than 10% as much cell death in cells expressing the modified CD117 polypeptide as compared to in cells expressing only the wild-type CD117 polypeptide. In some embodiments, the anti-CD117 antibody is selected from the group consisting of: JSP191, CDX-0159, AB85, and FSI-174.

In a further related embodiment, the disclosure provides a pharmaceutical composition comprising the modified cells, e.g., HSCs, comprising the nucleic acid encoding the modified CD117 polypeptide, and a pharmaceutically acceptable excipient, carrier, or diluent. In some embodiments, the pharmaceutical composition further comprises an anti-CD117 antibody. In certain embodiments, the pharmaceutical composition further comprises one or more anti-CD47, anti-CD40L, anti-CD122, anti-CD4, and/or anti-CD8 antibody.

I certain embodiments, the disclosure provides a pharmaceutical composition comprising a pharmaceutically acceptable excipient, carrier, or diluent and a modified hematopoietic stem cell (HSC) or a hematopoietic stern and progenitor cell (HSPC), wherein the modified HSC or HSPC comprises a modified CD117 polypeptide, optionally wherein the modified CD117 polypeptide has constitutive c-Kit signaling and/or kinase activity, and optionally wherein the modified cell is capable of proliferation and/or survival when contacted with an anti-c-Kit monoclonal antibody capable of inhibiting proliferation and/or survival of an HSPC expressing only a wild-type CD117. In certain embodiments, the c-Kit signaling and/or kinase activity of the modified CD117 is not substantially inhibited by an anti-c-Kit monoclonal antibody. In certain embodiments, the anti-c-Kit monoclonal antibody inhibits binding of SCF to CD117. In certain embodiments, the anti-c-Kit monoclonal antibody comprises one or more of the six CDRs present in any one of JSP191, AB85, CDX-0159, or FSI-174. In certain embodiments, the anti-c-Kit monoclonal antibody is any one of JSP191, AB85, CDX-0159, or FSI-174. In certain embodiments, the anti-c-Kit antibody is JSP191. In certain embodiments, the anti-c-Kit antibody is FSI-174. In certain embodiments, the modified CD117 comprises one or more amino acid modifications as compared to the wild-type CD117 polypeptide. In certain embodiments, the one or more amino acid modifications comprise one or more amino acid substitutions, insertions, or deletions. In certain embodiments, one or more of the amino acid modifications are present within surface exposed amino acid residues of the extracellular domain, within the membrane spanning domain, or within an intracellular domain of the modified CD117 polypeptide. In certain embodiments, the modified CD117 polypeptide comprises substitution or deletion of one or more of the following amino acids present in wild type human CD117: N505 or D816. In certain embodiments, the modified CD117 polypeptide comprises a D816V substitution and/or a N5051 substitution as compared to wild type human CD117. In certain embodiments, the modified CD117 polypeptide has at least 90%, at least 95%, at least 98%, or at least 99% sequence homology to wild type CD117 polypeptide. In certain embodiments, the wild type CD117 polypeptide is a wild type human CD117 polypeptide, optionally having one of the following amino acid sequences:

(SEQ ID NO: 1)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
or
(SEQ ID NO: 2)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNDSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV.

In certain embodiments, the modified cell expresses both the modified CD117 polypeptide and a wild type CD117 polypeptide. In certain embodiments, the modified cell expresses the modified CD117 polypeptide transiently. In certain embodiments, the HSC or HSPC is CD34+, optionally wherein the HSPC is CD34+/CD90+, CD34+/CD38−, or CD34+/CD38−/CD90+, or CD34+CD133+. In certain embodiments, the cell is a human cell. In certain embodiments, the cell was obtained from a mammalian donor. In certain embodiments, the mammalian donor is a subject in need of a hematopoietic cell transplant (HCT). In certain embodiments, the mammalian donor is a healthy donor. In certain embodiments, the cell obtained from the mammalian donor was modified ex vivo. In certain embodiments, the pharmaceutical composition further comprises an anti-CD117 antibody, an anti-CD47, anti-CD40L, anti-CD122, anti-CD4, and/or an anti-CD8 antibody.

In a related aspect, the disclosure includes a method of modifying a cell, e.g., an HSC, comprising introducing a nucleic acid or vector encoding a modified CD117 polypeptide into the cell, optionally wherein the cell is transiently modified, and optionally wherein the method is for preparing modified cells for hematopoietic cell transplantation (HCT) into a mammalian subject. In certain embodiments, the nucleic acid or vector is introduced into the cell by transfection, transduction, infection, electroporation, or nanopore technology.

In another aspect, the disclosure includes a method of treating a mammalian subject in need thereof, comprising administering to the subject a pharmaceutical composition comprising modified cells, e.g., HSCs, comprising the nucleic acid encoding the modified CD117 polypeptide and/or the modified CD117 polypeptide. In some embodiments, the method further comprises administering to the subject a conditioning regimen to facilitate or increase engraftment of the modified cells, or deplete endogenous, wild-type HSCs, wherein the conditioning regimen is administered prior to or concurrent with the administering of the pharmaceutical composition. In some embodiments, the conditioning regimen comprises or consists of an anti-CD117 antibody, optionally JSP191. In some embodiments, the conditioning regimen comprises one or more of: chemotherapy (optionally a nucleoside analog and/or an alkylating agent), monoclonal antibody therapy, and radiation, optionally radiation to the entire body. In particular embodiments, the conditioning regimen is milder than would be used if the subject was being administered hematopoietic stem cells that did not comprise the modified CD117 polypeptide. In some embodiments, the subject is not administered a conditioning regimen to facilitate or increase engraftment of the modified cells, prior to or concurrent with the administering of the pharmaceutical composition, or the conditioning regimen only comprises the anti-CD117 antibody. In particular embodiments, the method results in reduced toxicity, reduced morbidity, or reduced graft-versus-host disease, as compared to a method wherein a subject is administered hematopoietic stern cells that do not comprise the modified. CD117 polypeptide in combination with a conditioning regimen, e.g., a reduction of at least 10%, at least 20%, at least 30%, at least 40%, or at least 50% in toxicity, morbidity, and/or graft-versus-host disease.

In particular embodiments, the method is used to treat a disease or disorder selected from the group consisting of: a cancer, a cardiac disorder, a neural disorder, an autoimmune disease, an immunodeficiency, a metabolic disorder, and a genetic disorder. In certain embodiments, the cancer is a solid tissue cancer or a blood cancer, e.g., a leukemia, a lymphoma, or a myelodysplastic syndrome, such as acute myeloid leukemia (AML). In certain embodiments, the immunodeficiency is severe combined immunodeficiency (SCID). in certain embodiments, the genetic disorder is sickle cell disease or Fanconi anemia. In some embodiments, the methods further comprises administering to the subject another therapeutic agent for treatment of the disease or disorder.

### BRIEF DESCRIPTION OF THE DRAWINGS

FIG. 1 is a graph showing the OD595 of wild type BaF3 cells (BaF3), or BaF3 cells expressing wild-type CD117 (c-Kit) or the CD117-D816V mutant in the presence of the indicated concentrations of stem cell factor (SCF), and in the presence or absence of the anti-CD117 antibody, JSP191. These results show that the CD117-D816V mutant confers a proliferative advantage to cells, even in the absence of SCF and the presence of the anti-CD117 antibody, JSP191. At SCF (5 ng/ml), the lines of the graph from top to bottom are: D816V; D816V +JSP191; c-Kit; BaF3; and c-Kit+JSP191.

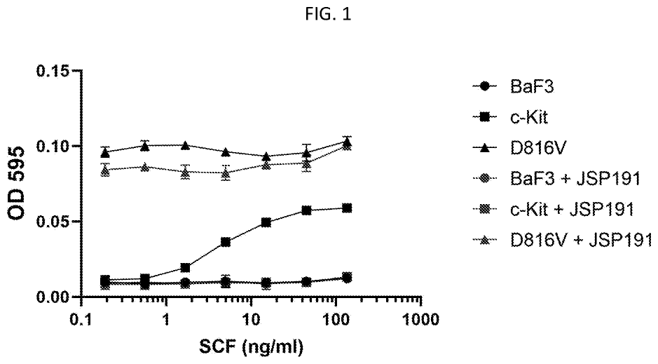

FIG. 2 is a table showing various myeloablative, reduced intensity myeloablative, and non-myeloablative conditioning regimens, reproduced from Atilla, Erden et al. “A Review of Myeloablative vs Reduced Intensity/Non-Myeloablative Regimens in Allogeneic Hematopoietic Stem Cell Transplantations.” Balkan Medical Journal, Vol. 34, 1 (2017):1-9. doi:10.4274/balkanmedj.2017.0055.

### DETAILED DESCRIPTION OF THE INVENTION

Hematopoietic stem cell transplantation (HCT) can be curative therapy for many diseases, based on the principle that healthy hematopoietic stem cells (HSCs) and/or hematopoietic stem and progenitor cells (HSPCs) replace abnormal HSCs. However, HCT is not widely used due to the toxicities associated with the current practices of this procedure. The deleterious effects of HCT include substantial tissue injury and even mortality from the use of chemotherapy and/or radiation prior to transplant (which are needed to prepare recipients to accept donor or autologous gene-corrected cells) and graft-vs-host disease (GVHD) caused by donor lymphocytes that are contained within allogeneic hematopoietic grafts. Despite the known complications caused by chemotherapy and/or radiation, and the infusion of donor lymphocytes in the allograft, these modalities are incorporated into HCTs, because they facilitate the engraftment of donor HSCs. Furthermore, HCTs can fail because donor HSC fail to engraft and/or fail to persist following the HCT procedure.

Certain HCT procedures include conditioning a patient prior to HCT by treatment with an anti-c-Kit antibody that inhibits stem cell factor (SCF) from binding to c-Kit on the surface of a patient's endogenous HSCs, which depletes endogenous HSCs prior to transplant of HSCs and/or HSPCs into the patient. However, this typically requires a significant washout period of about a week or more following administration of the anti-c-Kit antibody, so there are few remaining anti-c-Kit antibodies that would deplete the transplanted HSCs and/or HSPCs.

The present disclosure provides compositions and methods that augment the ability of donor or autologous gene-corrected HSCs and/or HSPCs to engraft and/or persist in recipients, thereby increasing the likelihood of success of an HCT procedure, and reducing the toxicities associated with HCT. In particular, the disclosure provides modified HSCs and/or HSPCs for transplant that comprise a modified CD117 polypeptide that has constitutive activity or retains activity (e.g., c-Kit kinase activity), even in the presence of anti-c-Kit antibodies (e.g., JSP191) that inhibit SCF binding to the modified CD117 polypeptide. In particular embodiments, the modified HSCs and/or HSPCs have activity (e.g., c-Kit kinase activity) even when not bound by SCF. In particular embodiments, the modified HSCs and/or HPSCs are not substantially depleted by anti-c-Kit antibodies, e.g., anti-c-Kit antibodies used in a conditioning regimen to deplete endogenous HSCs and/or HSPCs. Accordingly, the modified cells can be transplanted into the subject in the presence of the anti-c-Kit antibodies without being subject to depletion, thus providing an improved method of conditioning a patient for HCT and potentially allowing a reduced washout period and/or other advantages. The HCT methods provided herein result in a reduction in the need for intensive chemotherapy, radiation, and/or donor lymphocytes or other cells used to facilitate HSC engraftment, thereby reducing the toxicity of HCT. Compositions and methods disclosed herein may be used to treat all disorders for which blood stem cell transplantation is indicated.

Binding of SCF and/or anti-c-Kit antibodies to the modified CD117 polypeptides may be determined by a variety of methods known in the art. For example, binding may be determined using transiently transfected HEK293T cells that express the modified CD117. Following transfection, cells are incubated with SCF and/or anti-c-Kit antibodies. Bound MAbs may be detected using an Alexa Fluor 488-conjugated secondary antibody and cellular fluorescence determined by flow cytometry. Bound SCF may be determined using a fluorescently-labeled antibody that binds SCF and flow cytometry.

In certain embodiments, the disclosure provides for compositions and methods for the ex vivo introduction of the CD117 variants and mutants (modified CD117), by RNA-based and/or DNA-based methods, into HSCs and/or HSPCs, including but not limited to CD34+ cells or subsets of CD34+ cells, such that the HSCs and/or HSPCs are able to be successfully transplanted into recipients. The modified CD117 may be expressed transiently in the modified HSCs and/or HSPCs. For example, a nucleic acid encoding a modified CD117 may be transiently introduced into HSCs/and/or HSPCs prior to transplant, where it expresses the modified CD117. Thus, the modified CD117 may be expressed in addition to the endogenous wild type CD117. Transplantation of these modified HSCs may be done after or in combination with conditioning therapies, including treatment with antibodies (such as anti-CD117 antibodies). These HSCs may be transplanted alone or in combination with other cells.

It is to be understood that this invention is not limited to the particular methodology, products, apparatus and factors described, as such methods, apparatus and formulations may, of course, vary. It is also to be understood that the terminology used herein is for the purpose of describing particular embodiments only, and is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

As used herein, “antibody” includes reference to an immunoglobulin molecule immunologically reactive with a particular antigen, and includes both polyclonal and monoclonal antibodies. The term also includes genetically engineered forms such as humanized antibodies, chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

The assignment of amino acids to each VL and VH domain (and the CDRs therein) is in accordance with any conventional definition of CDRs. Conventional definitions include: the Kabat definition (Kabat, Sequences of Proteins of Immunological Interest (National Institutes of Health, Bethesda, Md., 1987 and 1991); the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering) in which corresponding residues between different heavy chains or between different light chains are assigned the same number. Unless otherwise specified numbering of positions within the variable regions of antibodies is Kabat numbering. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR H1 is a composite Chothia-Kabat CDR H1 and other CDRs contain Kabat CDR residues and no additional CDR residues based on other definitions.

The term “polynucleotide” refers to a polymeric form of nucleotides of any length, including deoxyribonucleotides or ribonucleotides, or analogs or mixtures thereof. A polynucleotide may comprise modified nucleotides, such as methylated nucleotides and nucleotide or nucleoside analogs, and may be interrupted by non-nucleotide components. If present, modifications to the nucleotide structure may be imparted before or after assembly of the polymer. The term polynucleotide, as used herein, includes, but is not limited to, double- and single-stranded molecules, and mixtures thereof. Unless otherwise specified or required, any embodiment of the invention described herein that is a polynucleotide encompasses both the double-stranded form and each of two complementary single-stranded forms known or predicted to make up the double-stranded form, whether as RNA or DNA, or a mixture thereof.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

A polynucleotide or polypeptide has a certain percent “sequence identity” to another polynucleotide or polypeptide, meaning that, when aligned, that percentage of bases or amino acids are the same when comparing the two sequences. The term “sequence identity” refers to the percentage identity of a polypeptide or polynucleotide sequence of interest to a reference sequence, calculated as 100 times the number of exact matches in an optimal alignment of the sequence of interest to the reference sequence divided by the total length of the reference sequence (including gaps). When comparing DNA and RNA sequences, thymine (T) and uracil (U) are counted as a match.

Sequence similarity can be determined in a number of different manners. To determine sequence identity, sequences can be aligned using the methods and computer programs, including BLAST, available over the worldwide web at ncbi.nlm.nih.gov/BLAST/. Unless indicated to the contrary, sequence identity is determined using the BLAST algorithm (e.g., bl2seq) with default parameters. An optimal alignment of the sequences may be generated using the European Molecular Biology Open Software Suite (EMBOSS) needle program available at www.ebi.ac.uk, as described in Maderia et al. Nucleic Acids Res. 47(W1): W636-W641 (2019). Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors, liposomes, and other gene delivery vehicles.

An “expression vector” as used herein encompasses a vector, e.g., plasmid, minicircle, viral vector, liposome, and the like, as discussed herein or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g., promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues, and species, or it may be cell-type specific, tissue-specific, or species specific. Promoters may be “constitutive,” meaning continually active, or “inducible,” meaning the promoter can be activated or deactivated by the presence or absence of biotic or abiotic factors.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “native” or “wild-type” as used herein refers to a nucleotide sequence, e.g., gene, or gene product, e.g., RNA or polypeptide, that is present in a wild-type cell, tissue, organ or organism, e.g., a wild-type human gene or protein sequence. The term “variant” as used herein refers to a mutant of a reference polynucleotide or polypeptide sequence (e.g., a native or wild-type) polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full-length native polynucleotide sequence. As another example, a variant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference sequence, e.g., a native sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence. In particular embodiments, modified CD117 polypeptides comprises a modification as disclosed herein and a deletion, such as an N-terminal and/or C-terminal deletion, yet substantially retain CD117 activity, e.g., kinase activity. In particular embodiments of any modified CD117 polypeptide disclosed herein, the modified CD117 has constitutive kinase activity.

The term “stem cell” as used herein refers to a mammalian cell that has the ability both to self-renew, and to generate differentiated progeny (see Morrison et al. (1997) Cell 88:287-298). Endogenous stem cells may be characterized by the presence of markers associated with specific epitopes. Hematopoietic stem cells (HSC) are multipotent cells that reside in the bone marrow (BM) and are responsible for the life-long production of mature blood cells. Hematopoietic stem and progenitor cells (HSPCs) include HSCs as well as hematopoietic progenitor cells that reside in bone marrow and are capable of differentiating into mature blood cells. In some embodiments, HSC and/or HSPC engraftment cells may be fresh, frozen, or subject to prior culture. HSC and/or HSPC may be obtained from fetal liver, bone marrow, cord blood, or peripheral blood, by a donor (allogeneic), the patient themselves (autologous), or any other conventional source.

The terms “administering” or “introducing” or “providing”, as used herein, refer to delivery of a composition to a cell, to cells, tissues and/or organs of a subject, or to a subject. Such administering or introducing may take place in vivo, in vitro, or ex vivo.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered before or during the symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

As used herein, the term “substantially” means by a significant or large amount or degree. For example, to “substantially” increase may mean to increase by at least two-fold, at least three-fold, at least four-fold, at least five-fold, or at least ten-fold, and to “substantially” decrease may mean to decrease by at least 30%, at least 40%, at least 50%, at least 60%, at least 70%, at least 80%, or at least 90%.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### CD117 Variant Polypeptides and Polynucleotides

CD117, also known as c-kit or stem cell factor receptor (SCFR), has a molecular weight of 145 kDa as a mature protein and is a member of the type III receptor tyrosine kinase (RTK) family that includes platelet-derived growth factor (PDGF) receptors and the macrophage colony-stimulating factor 1 (CSF-1) (c-fms) receptor. CD117 is essential for the development of normal hematopoietic cells and plays an important role in the survival, proliferation, and differentiation of mast cells, melanocytes, and germ cells. It is expressed by hematopoietic cells in the embryonic liver throughout development, and by more committed progenitors, such as myeloid, erythroid, megakaryocytic, natural killer, and dendritic progenitor cells.

CD117 includes: an approximately 519 amino acid extracellular domain comprised of five immunoglobulin-like domains; a transmembrane segment; a juxtamembrane domain; and a protein kinase domain that contains an insert of about 80 amino acid residues. Approximately 184 amino acids of the extracellular domain are surface exposed, which were identified based on x-ray crystallographic studies. The crystallographic structure of CD117 is provided in, e.g., Mol, et al., Accelerated Publications, Volume 278, ISSUE 34, P31461-31464, Aug. 22, 2003; Ogg et al., RCSB Protein Data Bank, 6XV9, Crystal structure of the kinase domain of human c-KIT in complex with a type-II inhibitor, DOI: 10.2210/pdb6XV9/pdb; McAuley et al., RC SB Protein Data Bank Alkynyl Benzoxazines and Dihydroquinazolines as Cysteine Targeting Covalent Warheads and Their Application in Identification of Selective Irreversible Kinase Inhibitors, DOI: 10.1021/jacs.9b13391; Schimpl et al., RCSB Protein Data Bank 6GQM, Crystal structure of human c-KIT kinase domain in complex with a small molecule inhibitor, AZD3229, DOI: 10.1021/acs.jmedchem.8b00938; and Lin et al., RCSB Protein Data Bank Identification of a Multitargeted Tyrosine Kinase Inhibitor for the Treatment of Gastrointestinal Stromal Tumors and Acute Myeloid Leukemia, DOI: 10.1021/acs.jmedchem.9b01229. Binding of CD117 to its ligand (stem cell factor; SCF) induces receptor dimerization, trans autophosphorylation of the kinase domain, recruitment of signaling proteins via phosphotyrosine binding or Src homology 2 (SH2) domains, and subsequent signal transduction.

In one aspect, the disclosure provides a modified CD117 polypeptide comprising one or more amino acid modifications as compared to a wild type CD117 polypeptide. In particular embodiments, the one or more amino acid modifications comprise one or more amino acid substitutions, insertions, or deletions. In certain embodiments, the one or more amino acid modifications are located in the extracellular domain of the CD117 polypeptide. In certain embodiments, the one or more amino acid modifications are located in one or more surface exposed amino acids or regions of the CD117 polypeptide's extracellular domain. In certain embodiments, the modified CD117 comprises one or more modification within the juxtamembrane region or the kinase domain. In particular embodiments, the modified CD117 polypeptides comprise one or more deletions, e.g., an N-terminal or C-terminal deletion, optionally wherein the deletion does not substantially impair biological activity, e.g., signaling, of the modified CD117 polypeptide. In certain embodiments, the modified CD117 polypeptides retain or have at least 90%, at least 95%, at least 98%, or at least 99% sequence homology to the wild type CD117 polypeptide. In particular embodiments, the one or more amino acid modifications comprise one or more amino acid substitutions or deletions of an amino acid residue selected from the following in human CD117: N505 or D816. In certain embodiments, the one or more amino acid modifications comprise one or more amino acid substitutions, e.g., of any of these residues. In certain embodiments, the amino acid residue is substituted by any other amino acid, by alanine. In certain embodiments, the amino acid substitution is a conservative amino acid substitution. The term “conservative substitution” as used herein denotes that one or more amino acids are replaced by another, biologically similar residue.

In the scheme below, conservative substitutions of amino acids are grouped by the indicated physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by the indicated physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

In particular embodiments, the one of more amino acid substitutions comprises a D816V substitution and/or a N505I substitution.

In certain embodiments, the wild type CD117 polypeptide upon which the variant is based is a human CD117 polypeptide, while in other embodiments, it is another mammalian CD117 polypeptide. Sequences of human and mammalian CD117 polypeptides are known in the art. Due to alternative splicing of the c-kit gene, the human CD117 polypeptide is expressed as various isoforms, and any of these may be used according to the disclosure. These isoforms include two GNK+ and GNNK− isoforms (also denoted c-Kit and c-KitA, respectively), which differ by the presence or absence of four amino acids, 510-GNNK-513 (SEQ ID NO: 25) in the extra-cellular domain adjacent to the trans-membrane domain, and which are coexpressed in most tissues, although the GNNK− isoform usually predominates. Isoforms may also differ in the presence or absence of a Ser residue at position 715 in the inter-kinase domain, and the disclosure also includes isoforms of CD117, including those shown below, in which Ser175 is either present or absent. These isoforms may comprise any of the modifications disclosed herein, including, e.g., an N505I or D816V modification, and variants thereof, e.g., comprising at least 90%, at least 95%, at least 98%, or at least 99% sequence identity thereto.

In particular embodiments, the wild type CD117 polypeptide is the GNNK+ or GNNK− isoform and comprises or consists of one of the following amino acid sequences (the GNNK tetrapeptide (SEQ ID NO: 25), and the N505 and D816 residues are in bold; numbering is based on GNNK− isoform):

(SEQ ID NO: 1)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
or
(SEQ ID NO: 2)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNDSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV.

In certain embodiments, the modified CD117 polypeptide comprises or consists of either of the following sequences:

(SEQ ID NO: 3)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNVSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
(SEQ ID NO: 4)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNVSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV;
(SEQ ID NO: 5)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFIFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
or
(SEQ ID NO: 6)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFIFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNDSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV,

or a variant or fragment thereof, e.g., having at least 90%, at least 95%, at least 98%, or at least 99% identity thereto. In particular embodiments, the variant retains the N505I or D816V amino acid substitution present in the modified CD117. In certain embodiments, the CD117 variant comprises a different amino acid modification that confers constitutive activity to the modified CD117. In particular embodiments, a fragment substantially retains CD117 kinase activity, e.g., retains at least 50% CD117 kinase activity as.

In certain embodiments, the modified CD117 polypeptide substantially retains kinase activity as compared to the wild type CD117 polypeptide. In particular embodiments, the modified CD117 polypeptide has at least 50%, at least 60%, at least 70%, at least 80%, at least 90%, or 100% of the kinase activity of the wild type CD117 polypeptide when bound by SCF, and in certain embodiments, the modified CD117 has this activity even in the absence of SCF binding. In some embodiments, the modified CD117 polypeptide has increased kinase activity as compared to the wild type CD117 polypeptide. In particular embodiments, the modified CD117 polypeptide has at least 150%, at least 200%, at least 300%, at least 500%, at least 750%, or at least 1000% of the kinase activity of the wild type CD117 polypeptide. In particular embodiments, the modified CD117 has constitutive kinase activity, even in the absence of SCF binding or in the presence of the anti-c-Kit antibody. Kinase activity may be determined using assays known in the art, including the ADP-Glo™ Kinase Assay, which is a luminescent kinase assay that measures ADP formed from a kinase reaction; ADP is converted into ATP, which is converted into light by Ultra-Glo™ Luciferase (available from Promega Corporation, Madison, WI). In certain embodiments, the modified CD117 polypeptide constitutively phosphorylates Gab2, Shc, SILIP-2 and/or Cbl.

In particular embodiments, the one or more amino acid modifications do not substantially inhibit or reduce binding of stem cell factor (SCF) to the modified CD117 polypeptide when expressed in cells, as compared to the binding of SCF to the wild type CD117 polypeptide. In particular embodiments, the one or more amino acid modifications do not substantially inhibit or reduce binding of stem cell factor (SCF) to the modified CD117 polypeptide expressed in cells as compared to the wild type CD117 polypeptide.

In certain embodiments, the modified CD117 is a modified CD117 having constitutive signaling or kinase activity, e.g., without bound SCF ligand. In certain embodiments, the modified CD117 has constitutive autophosphorylation activity, e.g., without bound SCF. A variety of such modified CD117 have been identified, e.g., in cancer cells, and any of these may be used according to the compositions and methods disclosed herein. Illustrative examples of activating or gain-of-function CD117 modifications include, but are not limited to, N505I, V559D, D816V, D816H, V568F, V570F, or Y703F, modifications or mutation of amino acid residues corresponding to 505, 522, 816, 557, 558, 559, 568, 569, 570, 703, 816, or deletion of codon 579 (Asp). See, e.g., Akin and Metcalfe, Journal of Allergy and Clinical Immunology, Vol. 114, Issue 1, p 13-19, Jul. 1, 2004; Hirotakoji et al., Science 23, January 1998, Vol. 279, Issue 5350, pp. 577-580; Sanlorenzo et al., J Proteomics 2016 Jul. 20, 144: 140-147, and references cited in any of the aforementioned, all of which are hereby incorporated by reference in their entireties. In particular embodiments, the amino acid modification is in the region between the transmembrane and tyrosine kinase domains. Mutations causing constitutive activation of c-Kit have been shown to be causative in some forms of mastocytosis, and several types of mutations have been associated with myeloproliferative disorders (MPDs), acute myelogenous leukemia (AML), sinonasal lymphomas, and gastrointestinal strornal tumors (GIST). These may be considered activating mutation of two types—‘regulatory type’ mutations, which affect regulation of the kinase molecule, and ‘enzymatic pocket type’ mutations, which alter the amino acid sequence directly forming the enzymatic site. Either type of mutation may be used according to various embodiments of the disclosure, including any of those disclosed in Longley et al., Leukemia Research, Vol. 25, Issue 7, July 2001, pp. 571-576, and references cited therein, all of which are incorporated herein by reference in its entireties.

In particular embodiments, the one or more amino acid modifications do not result in cells expressing only the modified CD117 having substantially inhibited or reduce c-Kit signaling or proliferation, optionally in response to SCF binding, as compared to the signaling in cells only expressing the wild type CD117 polypeptide. In particular embodiments, the modified CD117 retains at least 40%, at least 50%, at least 60%, at least 70%, at least 80%, at least 90%, or at least 95% c-Kit signaling and/or proliferation, optionally in response to SCF binding, as compared to the corresponding wild type CD117. In particular embodiments, the modified CD117 retains at least 40%, at least 50%, at least 60%, at least 70%, at least 80%, at least 90%, or at least 95% c-Kit signaling and/or proliferation, in the absence of SCF binding, as compared to the corresponding wild type CD117.

In particular embodiments, the one or more amino acid modifications do not substantially inhibit or reduce binding of an anti-c-Kit antibody to the modified CD117 polypeptide expressed in cells as compared to the wild type CD117 polypeptide.

In particular embodiments, the anti-c-Kit antibody comprises the six CDRs present in any one of JSP191, AB85, CDX-0159, or FSI-174. In particular embodiments, the anti-c-Kit antibody in any one of JSP191, AB85, CDX-0159, or FSI-174.

In some embodiments, the anti-c-Kit antibody is JSP191 or comprises the six CDRs present in JSP191.

In some embodiments, the anti-c-Kit antibody is AB85 or comprises the six CDRs present in AB85.

In some embodiments, the anti-c-Kit antibody is CDX-0159 or comprises the six CDRs present in CDX-0159.

In some embodiments, the anti-c-Kit antibody is FSI-174 or comprises the six CDRs present in FSI-174.

The disclosure also provides nucleic acid or polynucleotides encoding a modified CD117 polypeptide disclosed herein. In particular embodiments, the nucleic acid comprises RNA, DNA, or a combination thereof, and in particular embodiments, the nucleic acid comprises single-stranded and/or double-stranded regions, or a mixture thereof In certain embodiments, the nucleic acid is a double-stranded DNA, and in certain embodiments, the nucleic acid is a single stranded RNA, e.g., a messenger RNA (mRNA). In certain embodiments, the nucleic acid comprises a modified mRNA. In particular embodiments, the polynucleotides described herein, e.g., modified mRNA, are codon-optimized, e.g., to enhance expression of the encoded polypeptide in a host cell.

In particular embodiments, polynucleotide variants comprise one or more modified nucleotide or nucleoside. Modified mRNAs comprising one or more modified nucleoside have been described as having advantages over unmodified mRNAs, including increase stability, higher expression levels and reduced immunogenicity. Non-limiting examples of modifications to mRNAs that may be present in the nucleic acids encoding the modified CD117 polypeptides are described, e.g., in PCT Patent Application Publication Nos. WO2011/130624, WO2012/138453, WO2013052523, WO2013151666, WO2013/071047, WO2013/078199, WO2012045075, WO2014081507, WO2014093924, WO2014164253, US Patent Nos: U.S. Pat. No. 8,278,036 (describing modified mRNAs comprising pseudouridine), U.S. Pat. No. 8,691,966 (describing modified mRNAs comprising pseudouridine and/or N1 -methylpseudouridine), U.S. Pat. No. 8,835,108 (describing modified mRNAs comprising 5-methylcytidine, U.S. Pat. No. 8,748,089 (describing modified mRNAs comprising pseudouridine or 1-methylpseudouridine). In particular embodiments, the modified mRNA comprises one or more nucleoside modification. In particular embodiments, the modified mRNA sequence comprises at least one modification as compared to an unmodified A, G, U or C ribonucleoside. For example, uridine can a similar nucleoside such as pseudouridine (Ψ) or N1-methyl-pseudouridine (m1Ψ), and cytosine can be replaced by 5-methylcytosine. In particular embodiments, the at least one modified nucleosides include N1-methyl-pseudouridine and/or 5-methylcytidine. In certain embodiments, all uridines in the modified mRNA are replaced with a similar nucleoside such as pseudouridine (Ψ) or N1-methyl-pseudouridine (m1Ψ), and/or all cytosines in the modified tri RNA are substituted with a similar nucleoside such as 5-methylcytosine. In particular embodiments, the modified mRNA comprises a 5′ terminal cap sequence followed by a sequence encoding the modified CD117 polypeptide, followed by a 3′ tailing sequence, such as a polyA or a polyA-G sequence.

In certain embodiments, the nucleic acid, e.g., a modified mRNA, is associated with one or more lipids, e.g., to facilitate delivery across the cell membrane, shield its negative charge, and/or to protect against degradation by nucleases. In certain embodiments, the nucleic acid is associated with or present within a lipid nucleic acid particle, a lipid nanoparticle, or a liposome. In certain embodiments, the lipid nucleic acid particle, a lipid nanoparticle, or a liposome facilitates delivery or uptake of the nucleic acid by a cell. In certain embodiments, mRNA, optionally modified mRNA, is co-formulation into lipid nanoparticles (LNPs). In particular embodiments, mRNA-LNP formulations comprise: (1) an ionizable or cationic lipid or polymeric material bearing tertiary or quaternary amines to encapsulate the polyanionic mRNA; (2) a zwitterionic lipid (e.g., 1,2-dioleoyl-sn-glycero-3-phosphoethanolamine [DOPE]) that resembles the lipids in the cell membrane; (3) cholesterol to stabilize the lipid bilayer of the LNP; and (4) a polyethylene glycol (PEG)-lipid to lend the nanoparticle a hydrating layer, improve colloidal stability, and reduce protein absorption.

In certain embodiments, the nucleic acid encoding the modified CD117 polypeptide is present in a vector. In particular embodiments, the vector is capable of delivering the nucleic acid into mammalian HSCs or other stem cells, e.g., into the nucleus of the HSCs or stem cells. In certain embodiments, the vector is an episomal vector, e.g., a plasmid. In particular embodiments, the vector is an expression vector comprising a promoter sequence operatively linked to a nucleic acid sequence encoding the modified CD117 polypeptide. In particular embodiments, the expression vector comprises a promoter sequence that facilitates expression of the encoded modified CD117 polypeptide in HSCs or other stem cells. In particular embodiments, the expression vector comprises 5′ and/or 3′ cellular or viral UTRs or the derivatives thereof upstream and downstream, respectively, of the sequence encoding the modified CD117 polypeptide.

In certain embodiments, the vector is a viral vector, optionally an AAV vector, a cytomegalovirus vector, an adenovirus vector, or a lentiviral vector. In certain embodiments, a viral vector infects an HSC when viral vector and the HSCs are incubated together for at least about 24 hours in a culture medium.

### Modified Hematopoietic Stem Cells and Pharmaceutical Compositions

In a related aspect, the disclosure provides modified cells, e.g., HSCs and/or HSPCs, comprising a nucleic acid encoding a modified CD117 polypeptide described herein. In certain embodiments, the modified CD117 polypeptide comprises one or more amino acid substitutions, e.g., at one or more of the following amino acids present in wild type human CD117: N505 or D816. In particular embodiments, the modified CD117 polypeptide comprises a D816V substitution and/or a N505I substitution.

In certain embodiments, the nucleic acid encoding the modified CD117 polypeptide is transiently present in the modified cell, and it is not present within the genome of the cell. In particular embodiments, the modified cell expresses and/or comprises the modified CD117 polypeptide, and in particular embodiments, the modified CD117 polypeptide is present on the cell surface, e.g., with the extracellular domain present outside the modified cell. In certain embodiments, the modified cell is transduced with or infected with an expression vector, optionally a viral vector. In particular embodiments, the modified cell expresses and/or comprises both the modified CD117 polypeptide and a wild type, endogenous CD117 polypeptide, and in particular embodiments, both the modified CD117 polypeptide and the wild type, endogenous CD117 polypeptide are present on the cell surface, e.g., with their extracellular domains present outside the modified cell. In certain embodiments, the modified CD117 polypeptide is present on the cell surface, e.g., with its extracellular domain present outside the modified cell. In particular embodiments, the CD117s are human CD117 or modified forms thereof.

In certain embodiments, the modified cell comprising a modified CD117 polypeptide and/or encoding nucleic acid is a host cell, such as, e.g., an HEK293 cell that may be used to produce modified CD117 polypeptides. In preparing the subject compositions, any host cells may be employed, including but not limited to, for example, mammalian cells (e.g., 293 cells), insect cells (e.g., SF9 cells), microorganisms, and yeast.

In particular embodiments, the modified cell is a stem cell or pluripotent cell, and in certain embodiments, the stem cell is a hematopoietic stem cell (HSC) or an HSPC. In some embodiments, the stem cell is a mammalian cell that has the ability both to self-renew, and to generate differentiated progeny. In certain embodiments, the stem cell is a human cell. The stem cell may have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages.

Hematopoietic stem cells (EISCs) are maintained throughout life (self-renewing). They produce hematopoietic progenitor cells that differentiate into every type of mature blood cell within a well-defined hierarchy. Hematopoietic stem cells can also be generated in vitro, for example from pluripotent embryonic stem cells, induced pluripotent cells, and the like. For example, see Sugimura et al. (2017) Nature 545:432-438, herein specifically incorporated by reference, which details a protocol for generation of hematopoietic progenitors.

The cells may be fresh, frozen, or have been subject to prior culture. They may be fetal, neonate, adult, etc. Hematopoietic stem cells and HSPCs may be obtained from fetal liver, bone marrow, blood, particularly G-CSF or GM-CSF mobilized peripheral blood, or any other conventional source. Cells for engraftment are optionally isolated from other cells, where the manner in which the stem cells are separated from other cells of the hematopoietic or other lineage is not critical to this invention. If desired, a substantially homogeneous population of stem or progenitor cells may be obtained by selective isolation of cells free of markers associated with differentiated cells, while displaying epitopic characteristics associated with the stem cells.

Modified HSCs may be produced using HSCs obtained from a mammalian donor. In particular embodiments, the donor is a subject in need of a hematopoietic stem cell transplant, e.g., a subject diagnosed with a disease or disorder that can be treated with HCT. In other embodiments, the modified HSCs may be produced using HSCs obtained from a healthy donor, e.g., wherein the modified HSCs are to be used to treat a different subject with HCT. Thus, the modified HSCs may be autologous or allogeneic to a subject in need for HCT.

Prior to harvesting stem cells from a donor, the bone marrow can be primed with granulocyte colony-stimulating factor (G-CSF; filgrastim [Neupogen]) to increase the stem cell count. Mobilization of stem cells from the bone marrow into peripheral blood by cytokines such as G-CSF or GM-CSF has led to the widespread adoption of peripheral blood progenitor cell collection by apheresis for hematopoietic stem cell transplantation. The dose of G-CSF used for mobilization may be about 10 ug/kg/day. In autologous donors who are heavily pretreated, however, doses of up to about 40 ug/kg/day can be given. Mozobil may be used in conjunction with G-CSF to mobilize hematopoietic stem cells to peripheral blood for collection.

Among hematopoietic stem cell (HSC) markers, CD34 is well known for its unique expression on HSCs. In certain embodiments, the modified cell is a CD34+ cell. In particular embodiments, the modified cell is a subset of HSCs that has one of the following patterns or combinations of cell surface marker expression: CD34+/CD90+, CD34+/CD38−, or CD34+/CD38−/CD90+. The CD34+ and/or CD90+ cells may be selected by affinity methods, including without limitation magnetic bead selection, flow cytometry, and the like from the donor hematopoietic cell sample. The HSC composition may be at least about 50% pure, as defined by the percentage of cells that are CD34+ in the population, may be at least about 75% pure, at least about 85% pure, at least about 95% pure, or more.

In certain embodiments, the hematopoietic stem cells and/or HSPCs are obtained from bone marrow, peripheral blood, or umbilical cord blood and subsequently modified by introduction of the nucleic acid encoding the modified CD117 polypeptide into the cell. For example, the nucleic acid may be introduced by transfection or infection with a viral vector, or by contact with an mRNA.

In certain embodiments, the disclosure provides a method of modifying cells, including stem cells such as HSCs and/or HSPCs, comprising introducing the nucleic acid encoding a modified CD117 polypeptide into the cell. In particular embodiments, the introduced nucleic acid is present within a viral vector. In certain embodiments, the nucleic acid is associated with or present in a lipid nanoparticle, liposome, or the like. In certain embodiments, the nucleic acid remains present in the modified cell only transiently, or the nucleic acid only transiently expresses the modified CD117 polypeptide in the cell. In certain embodiments, the method is used to prepare modified cells for HCT treatment of a mammalian subject. In particular embodiments, the nucleic acid or vector may be introduced into the cell by a variety of methods known in the art, such as transfection, transduction, infection, electroporation, or nanopore technology. In particular embodiments, mRNA, e.g., modified mRNA is introduced into the cells using lipid nucleic acid particles (LNPs) or nanoparticles. Thus, cells, e.g., HSCs and/or HSPCS may be modified by introducing a nucleic acid encoding a modified CD117 polypeptide into the HSCs and/or HSPCs according to a variety of methods available in the art.

In particular embodiments, the modified cell expressing the modified CD117 polypeptide is not substantially inhibited, eliminated, depleted, or killed by monoclonal antibodies (mAbs) that bind endogenous or wild-type cell-surface CD117 and inhibit proliferation of or kill a cell expressing only the wild-type CD117 and not a modified CD117 polypeptide disclosed herein. in certain embodiments, proliferation of the modified cell expressing the modified CD117 polypeptide is inhibited, eliminated, depleted, or killed by less than 50%, less than 40%, less than 30%, less than 20%, or less than 10%, as compared to proliferation of the same cell type that is not modified, e.g., only expresses wild-type CD117.

Compositions and methods disclosed herein may be applicable to any anti-c-Kit antibody, particularly monoclonal anti-human c-Kit antibodies. Illustrative anti-c-Kit antibodies include, but are not limited to, SR-1, JSP191, 8D7, K45, 104D2, CK6, YB5.B8, AF-2-1, AF11, AF12, AF112, AF-3, AF-1-1, NF, NF-2-1, NF11, NF12, NF112, NF-3, HF11, HF12, and HF112. A number of antibodies contemplated by the disclosure that specifically bind human CD117 are known in the art and commercially available, including without limitation SR1, 2B8, ACK2, YB5-B8, 57A5, 104D2, etc. In certain embodiments, the anti-CD117 antibody is selected from the group consisting of: JSP191 (Jasper Therapeutics; Redwood City, CA); CDX-0159 (Celldex Therapeutics, Hampton, NJ); MGTA-117 (AB85) (Magenta Therapeutics, Cambridge, MA); CK6 (Magenta Therapeutics, Cambridge, MA); AB249 (Magenta Therapeutics, Cambridge, MA); and FSI-174 (Gilead, Foster City, CA). Antibodies from Magenta Therapeutics contemplated by the disclosure include but are not limited to those that are disclosed in US Patent Application Publication No. 20190153114, PCT Application Publication Nos. WO2019084064, WO2020/219748, and WO2020/219770. The FSI-174 antibody is disclosed in PCT application Publication No. WO2020/112687 and U.S. Patent Application Publication No. 20200165337. The disclosure includes but is not limited to any anti-c-Kit antibodies and/or CDR sets disclosed in any of the patent application disclosed herein, which are all incorporated by reference in their entireties.

In certain embodiments, the anti-c-Kit antibody binds to the extracellular region of CD117, i.e., amino acids 26-524. The sequence of this region is shown below:

(SEQ ID NO: 7)
QPSVSPGEPSPPSIHPGKSDLIVRVGDEIRLLCTDPGFVKWTFEILDETN
ENKQNEWITEKAEATNTGKYTCTNKHGLSNSIYVFVRDPAKLFLVDRSLY
GKEDNDTLVRCPLTDPEVTNYSLKGCQGKPLPKDLRFIPDPKAGIMIKSV
KRAYHRLCLHCSVDQEGKSVLSEKFILKVRPAFKAVPVVSVSKASYLLRE
GEEFTVTCTIKDVSSSVYSTWKRENSQTKLQEKYNSWHHGDFNYERQATL
TISSARVNDSGVFMCYANNTFGSANVTTTLEVVDKGFINIFPMINTTVFV
NDGENVDLIVEYEAFPKPEHQQWIYMNRTFTDKWEDYPKSENESNIRYVS
ELHLTRLKGTEGGTYTFLVSNSDVNAAIAFNVYVNTKPEILTYDRLVNGM
LQCVAAGFPEPTIDWYFCPGTEQRCSASVLPVDVQTLNSSGPPFGKLVVQ
SSIDSSAFKHNGTVECKAYNDVGKTSAYFNFAFKGNNKEQIHPHTLFTP.

In particular embodiments, the antibody is the humanized form of SR1, which is a murine anti-c-Kit antibody disclosed in U.S. Pat. Nos. 5,919,911 and 5,489,516. The humanized antibody, referred to as JSP191 (formerly referred to as AMG191), is described in U.S. Pat. Nos. 8,436,150, 8,791,249, and 7,915,391, and U.S. Patent Application Publication No. 20110223165. JSP191 is an aglycosylated IgG1 humanized antibody. JSP191 is a humanized monoclonal antibody in clinical development as a conditioning agent to clear hematopoietic stem cells from bone marrow. JSP191 specifically binds to human CD117, a receptor for stem cell factor (SCF), which is expressed on the surface of hematopoietic stem and progenitor cells (HSPCs). JSP191 blocks SCF from binding to CD117 and disrupts critical survival signals, leading to the depletion of hematopoietic stem cells.

The sequences of the heavy chains and light chains of JSP191 are disclosed as SEQ ID NO: 4 in U.S. Pat. No. 8,436,150 and SEQ ID NO: 2 in U.S. Pat. No. 8,436,150, respectively. The sequences of the heavy and light chains of JSP191 are:

Heavy Chain:
(SEQ ID NO: 8)
MDWTWRVFCLLAVAPGAHSQVQLVQSGAEVKKPGASVKVSCKASGYTFTS
YNMHWVRQAPGQGLEWMGVIYSGNGDTSYNQKFKGRVTITADKSTSTAYM
ELSSLRSEDTAVYYCARERDTRFGNWGQGTLVTVSSASTKGPSVFPLAPS
SKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDG
VEVHNAKTKPREEQYQSTYRVVSVLTVLHQDWLNGKEYKCKVSNKALPAP
IEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEA
LHNHYTQKSLSLSPGK
Light Chain:
(SEQ ID NO: 9)
MVLQTQVFISLLLWISGAYGDIVMTQSPDSLAVSLGERATINCRASESVD
IYGNSFMHWYQQKPGQPPKLLIYLASNLESGVPDRFSGSGSGTDFTLTIS
SLQAEDVAVYYCQQNNEDPYTFGGGTKVEIKRTVAAPSVFIFPPSDEQLK
SGTASVVCLLNNFYPREAKVQWKVDNALQSGNSQESVTEQDSKDSTYSLS
STLTLSKADYEKHKVYACEVTHQGLSSPVTKSFNRGEC

In certain embodiments, the variable heavy domain of JSP191 comprises the following sequence:

(SEQ ID NO: 10)
QVQLVQSGAEVKKPGASVKVSCKASGYTFTSYNMHWVRQAPGQGLEWMGV
IYSGNGDTSYNQKFKGRVTITADKSTSTAYMELSSLRSEDTAVYYCARER
DTRFGNWGQGTLVTVSS.

In certain embodiments, the variable light chain domain of JSP191 comprises the following sequence:

(SEQ ID NO: 11)
DIVMTQSPDSLAVSLGERATINCRASESVDIYGNSFMHWYQQKPGQPPKL
LIYLASNLESGVPDRFSGSGSGTDFTLTISSLQAEDVAVYYCQQNNEDPY
TFGGGTKVEIK.

The CDRs present in JSP191 are as follows: VH CDR1=YNMH (SEQ ID NO: 26); VH CDR2=IYSGNGDTSYNQKFKG (SEQ ID NO: 27); VH CDR3=ERDTRFGN (SEQ ID NO: 28); VL CDR1=RASESVDIYGNSFMH (SEQ ID NO: 29); VL CDR2=LASNLES (SEQ ID NO: 30); and VL CDR3=QQNNEDPYT (SEQ ID NO: 31).

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization. CDX-0159 and other anti-c-Kit antibodies are described in U.S. Pat. No. 10,781,267, and in particular embodiments, an anti-c-Kit disclosed herein comprises the CDRs of any of the antibodies disclosed therein. In certain embodiments, the anti-c-Kit antibody comprises: (i) a light chain variable region (“VL”) comprising the amino acid sequence:

DIVMTQSPSX_{K1}LSASVGDRVTITCKASQNVRTNVAWYQQKPGKAPKX_{K2}LIYSASYRYS GVPDRFX_{K3}GSGSGTDFTLTISSLQX_{K4}EDFAX_{K5}YX_{K6}CQQYNSYPRTFGGGTKVEIK (SEQ ID NO:12), wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain, and X_{K6} is an amino acid with an aromatic side chain; and (ii) a heavy chain variable region (“VH”) comprising the amino acid sequence: QVQLVQSGAEX_{H1}KKPGASVKX_{H2}SCKASGYTFTDYYINAVVX_{H3}QAPGKGLEWIARTYP GSGNTYYNEKFKGRX_{H4}TX_{H5}TAX_{H6}KSTSTAYMX_{H7}LSSLRSEDXFNAVYFCARGVYYFD YWGQGTTVTVSS (SEQ ID NO:13), wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In specific aspects, described herein are antibodies (e.g., human or humanized antibodies), including antigen-binding fragments thereof, comprising:
(i) VH CDRs of a VH domain comprising the amino acid sequence:

(SEQ ID NO: 14)
QVQLKQSGAELVRPGASVKLSCKASGYTFTDYYINWVKQRPGQGLEWIAR
IYPGSGNTYYNEKFKGKATLTAEKSSSTAYMQLSSLTSEDSAVYFCARGV
YYFDYWGQGTTLTVSS
or
(SEQ ID NO: 15)
QVQLKQSGAELVRPGASVKLSCKASGYTFTDYYINWVKQRPGQGLEWIAR
IYPGSGNTYYNEKFKGKATLTAEKSSSTAYMQLSSLTSEDSAVYFCARGV
YYFDYWGQGTTLTVSA,

and
(ii) VL CDRs of a VL domain comprising the amino acid sequence

(SEQ ID NO: 16)
DIVMTQSQKFMSTSVGDRVSVTCKASQNVRTNVAWYQQKPGQSPKALIYS
ASYRYSGVPDRFTGSGSGTDFTLTISNVQSEDLADYFCQQYNSYPRTFGG
GTKLEIKR.

MGTA-117 (AB85) is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells, and this antibody and others contemplated by the disclosure are described in U.S. Application No. 20200407440 and/or PCT Application No. WO2019084064. Epitope analysis of AB85 binding to CD177 is described in PCT Application Publication No. WO2020219770, which identified the following two epitopes within CD117:

| | (amino acids 60-90;) |
|---|---|
| (SEQ ID NO: 17) | |
| | EKAEATNTGKYTCTNKHGLSNSIYVFVRDPA, |
| | and |
| | (amino acids 100-130;) |
| (SEQ ID NO: 18) | |
| | RCPLTDPEVTNYSLKGCQGKP. |

The sequences of the variable heavy chain and variable light chains of AB85 are disclosed as SEQ ID NO: 143 and SEQ ID NO: 144 from PCT Application No. WO2019084064, respectively.

The heavy chain variable region (VH) amino acid sequence of AB85 is:

(SEQ ID NO: 19)
EVQLVQSGAEVKKPGESLKISCKGSGYSFTNYWIGWVRQMPGKGLEWMAI
INPRDSDTRYRPSFQGQVTISADKSISTAYLOWSSLKASDTAMYYCARHG
RGYEGYEGAFDIWGQGTLVTVSS.

The VH CDR amino acid sequences of AB85 are as follows: NYWIG (VH CDR1; SEQ ID NO: 32); IINPRDSDTRYRPSFQG (VH CDR2; SEQ ID NO: 33); and HGRGYEGYEGAFDI (VH CDR3; SEQ ID NO: 34).

The light chain variable region (VL) amino acid sequence of AB85 is:

(SEQ ID NO: 20)
DIQMTQSPSSLSASVGDRVTITCRSSQGIRSDLGWYQQKPGKAPKLLIYD
ASNLETGVPSRFSGSGSGTDFTLTISSLQPEDFATYYCQQANGFPLTFGG
GTKVEIK.

The VL CDR amino acid sequences of AB85 are as follows: RSSQGIRSDLG (VL CDR1; SEQ ID NO: 35); DASNLET (VL CDR2; SEQ ID NO: 36); and QQANGFPLT (VL CDR3; SEQ ID NO: 37).

FSI-174 is an anti-cKIT antibody being developed in combination with 5F9 as a non-toxic transplant conditioning regimen, as well as a treatment for targeted hematologic malignancies. The sequences of FSI-174 are disclosed in PCT Application Publication No. 2020/112687, U.S. Patent Application Publication No. 20200165337, and U.S. Pat. No. 11,041,022. In particular embodiments, an anti-c-Kit antibody comprises the three CDRs or variable heavy chain regions present in any of AH1, AH2, AH3, AH4, or AH5 disclosed therein, and/or the three CDRs or variable heavy chain regions present in any of AL1 or AL2 disclosed therein.

In certain embodiments, the CDRs present in FSI-174 and related antibodies are as follows: VH CDR1=SYNMH (SEQ ID NO: 38); VH CDR2=VIYSGNGDTSY(A/N)QKF(K/Q)G (SEQ ID NO: 39); VH CDR3=ERDTRFGN (SEQ ID NO: 40); VL CDR1=RAS(D/E)SVDIYG(N/Q)SFMH (SEQ ID NO: 41); VL CDR2=LASNLES (SEQ ID NO: 42); and VL CDR3=QQNNEDPYT (SEQ ID NO: 43). A/N and the like indicate that the amino acid position may be either of the two amino acids, in this example, A or N. In certain embodiments, CDRs present in the heavy variable region are CDRs H1, H2 and H3 as defined by Kabat: H1=SYNMH (SEQ ID NO: 38); H2=VIYSGNGDTSYAQKFKG (SEQ ID NO: 44); H3=ERDTRFGN (SEQ ID NO: 39); and the CDRs present in the light variable region are CDRs L1, L2 and L3 as defined by Kabat: L1=RASESVDIYGQSFM1-1 (SEQ ID NO: 45); L2=LASNLES (SEQ ID NO: 42); and L3=QQNNEDPYT (SEQ ID NO: 43), respectively except that 1, 2, or 3 CDR residue substitutions is/are present selected from N to A at heavy chain position 60, K to Q at heavy chain position 64 and N to Q at light chain position 30, positions being numbered according to Kabat. In certain embodiments, the antibody comprises any of the heavy chain variable region sequences (AH2, AH3, AH4) and/or light chain variable chain region sequences provided below (AL2), or the CDRs therein shown underlined:

AH2:
(SEQ ID NO: 21)
QVQLVQSGAEVKKPGASVKVSCKASGYTFTSYMNHWVRQAPGQGLEWMGV
IYSGNGDTSYAQKFKGRVTITADKSTSTAYMELSSLRSEDTAVYYCARER
DTREGNWGQGTLVTVSS
AH3:
(SEQ ID NO: 22)
QVQLVQSGAEVKKPGASVKVSCKASGYTFTSYMNHWVRQAPGQGLEWMGV
IYSGNGDTSYNQKFQGRVTITADKSTSTAYMELSSLRSEDTAVYYCARER
DTRFGNWGQGTLVTVSS
AH4:
(SEQ ID NO: 23)
QVQLVQSGAEVKKPGASVKVSCKASGYTFTSYMNHWVRQAPGQGLEWMGV
IYSGNGDTSYAQKFQGRVTITADKSTSTAYMELSSLRSEDTAVYYCARER
DTREGNWGQGTLVTVSS
AL2:
(SEQ ID NO: 24)
DIVMTQSPLSLPVTPGEPASISCRASESVDIYGQSFMHWYQQKPGQPPKL
LIYLASNLESGVPDRFSGSGSGTDFTLKISRVEAEDVGVYYCQQNNEDPY
TFGGGTKVEIK

In certain embodiments, the anti-CD117 antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-CD117 antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-CD117 antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-CD117 antibody comprises a heavy chain or variable region thereof comprising one, two, or three heavy chain CDRs disclosed herein, e.g., a JSP191 heavy chain. In particular embodiments, the anti-CD117 antibody comprises a light chain or variable region thereof comprising one, two, or three light chain CDRs disclosed herein, e.g., a JSP191 light chain.

In particular embodiments, the antibody binds to a region of wild-type CD117 or an epitope of wild-type CD117 that is modified in a modified CD117 polypeptides disclosed herein. In particular embodiments, the antibody does not bind a modified CD117 polypeptide disclosed herein, or binds to a modified CD117 polypeptide disclosed herein with reduced affinity, e.g., less than 50%, less than 25%, or less than 10%. Antibody affinity to a particular polypeptide, such as wild-type CD117 or a modified CD117 may be determined, e.g., by measuring the equilibrium dissociation constant between the antibody and its antigen (K_{D}), which may be determined by routine methods in the art, e.g., by surface plasmon resonance, as described in Hearty, Stephen, Paul Leonard and Richard O'Kennedy. “Measuring antibody—antigen binding kinetics using surface plasmon resonance.” Antibody Engineering: Methods and Protocols, Second Edition (2012): 411-442.

In particular embodiments, the modified cell expressing the modified CD117 polypeptide is capable of proliferating or surviving in the presence of an anti-CD117 antibody, e.g., an anti-CD117 antibody that blocks or inhibits binding of SCF to CD117 on the cell surface. In particular embodiments, proliferation and/or survival of the modified cell expressing the modified CD117 polypeptide, in the presence of an anti-CD117 antibody, e.g., an anti-CD117 antibody that blocks or inhibits binding of SCF to CD117 on the cell surface, is at least 90%, at least 80%, at least 70%, at least 60%, at least 50%, or at least 40% the level of proliferation and/or survival in the absence of the anti-CD118 antibody. In certain embodiments, the anti-CD117 antibody is capable of inhibiting proliferation of or inducing death or apoptosis of a cell expressing only the wild-type CD117 and not a modified CD117 polypeptide disclosed herein. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. Thus, in particular embodiments, the modified CD117 polypeptides disclosed herein, when expressed on a HSC and/or HSPC surface, are capable of substantially binding SCF in the presence of an anti-CD117 antibody that inhibit binding of SCF to endogenous, wild-type CD117 on the cell surface. Similarly, in particular embodiments, the modified CD117 polypeptides disclosed herein, when expressed on an HSC surface, are capable of intracellular signaling when bound by SCF, in the absence of and in the presence of an anti-CD117 antibody that inhibit binding of SCF to endogenous, wild-type CD117 on the cell surface. In particular embodiments, SCF binding and/or SCF-mediating signaling is in not substantially reduced in the presence of the anti-CD117 antibody, e.g., binding and/or signaling of the modified cell expressing the modified CD117 polypeptide is at least 40%, at least 50%, at least 60%, at least 70%, at least 80%, or at least 90% of the level of binding and/or signaling observed in the same cell type that is not modified, e.g., only expresses wild-type CD117.

c-Kit signaling or proliferation or viability may be determined using methods standard in the art. For example, in certain embodiments, c-Kit signaling or proliferation (e.g., in response to SCF), of cells comprising a modified CD117 polypeptide is determined using a cell line (e.g., Ba/F3 cells) engineered to express the modified CD117 polypeptide. Cells are cultured in the presence of IL-3, with or without stem cell factor (SCF), and in the presence or absence of an anti-CD117 antibody, e.g., JSP191. Control parental Ba/F3 cells do not proliferate in the absence of IL-3. Further, parental Ba/F3 cells do not express CD117 and are not responsive to SCF signaling. Proliferation in response to SCF binding may this be determined for cells overexpressing the modified CD117, e.g., in the presence and absence of the anti-CD117 antibody.

The disclosure also provides methods of preparing HSCs and/or HSPCs for HCT, comprising introducing a polynucleotide sequence encoding a modified CD117 described herein into the HSCs and/or HSPCs, In particular embodiments, the polynucleotide sequence encoding the modified CD117 is present within an mRNA or an expression vector, and the modified CD117 is transiently or constitutively expressed after it is introduced into the HSCs and/or HSPCs. The polynucleotide and/or vector may comprise nucleotide modifications, including any of those disclosed herein or known in the art, e.g., to increase expression or stability of the polynucleotide or vector.

For engraftment purposes, a composition comprising HSCs and/or HSPCs, is administered to a patient. Such methods are well known in the art. The stem cells are optionally, although not necessarily, purified. Abundant reports explore various methods for purification of stem cells and subsequent engraftment, including flow cytometry; an isolex system (Klein et al. (2001) Bone Marrow Transplant. 28(11):1023-9; Prince et al. (2002) Cytotherapy 4(2):137-45); immunomagnetic separation (Prince et al. (2002) Cytotherapy 4(2):147-55; Handgretinger et al. (2002) Bone Marrow Transplant. 29(9):731-6; Chou et al. (2005) Breast Cancer. 12(3):178-88); and the like. Each of these references is herein specifically incorporated by reference, particularly with respect to procedures, cell compositions and doses for hematopoietic stem cell transplantation.

The present disclosure also includes pharmaceutical compositions comprising one or more modified CD117 polypeptides, one or more polynucleotides or vectors comprising a sequence encoding a modified CD117 polypeptide (e.g., a modified mRNA), or a modified cell comprising a polynucleotide or vector encoding a modified CD117 polypeptide and/or expressing a modified CD117, in combination with one or more pharmaceutically acceptable diluent, carrier, or excipient.

The present invention discloses a pharmaceutical composition comprising a modified cell comprising a modified CD117 polypeptide (or nucleic acid sequence encoding the modified CD117 polypeptide) described herein and one or more pharmaceutically acceptable diluent, carrier, or excipient. In particular embodiments, the cell is a heterologous cell or an autologous cell obtained from the subject to be treated. In particular embodiments, the cell is a stem cell, e.g., a HSC and/or HSPC. In certain embodiments, the pharmaceutical composition further comprises one or more additional active agents. In certain embodiments, the one or more additional active agent comprises an anti-CD117 antibody. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. In certain embodiments, the one or more additional active agent comprises one or more anti-CD47, anti-CD40L, anti-CD122, anti-CD4, and/or anti-CD8 antibody.

The polynucleotides, polypeptides, and cells described herein can be combined with pharmaceutically-acceptable carriers, diluents and reagents useful in preparing a formulation that is generally safe, non-toxic, and desirable, and includes excipients that are acceptable for mammalian, e.g., human or primate, use. In certain embodiments, the pharmaceutical composition is a solution or suspension comprising modified cells disclosed herein. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile. For intravenous administration, suitable carriers include physiological saline, bacteriostatic water, or phosphate buffered saline (PBS). In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. In some cases, the composition is sterile and may be fluid to the extent that easy syringability exists. Prevention of the action of microorganisms can be achieved by various antibacterial and antifungal agents, for example, parabens, chlorobutanol, phenol, ascorbic acid, thimerosal, and the like. In certain embodiments, a pharmaceutical composition include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

### Methods of Use

In further aspects, the disclosure provides methods of treating a mammalian subject in need thereof, comprising administering to the subject modified cells, e.g., HSCs or HSPCs, comprising a modified CD117 polypeptide described herein and/or a nucleic acid encoding the modified CD117 polypeptide. In particular embodiments, the subject is in need of HCT or a hematopoietic stem cell transplant. The transplant may be autologous, allogeneic, or xenogeneic, including without limitation allogeneic haploidentical stem cells, mismatched allogeneic stem cells, genetically engineered autologous or allogeneic cells, etc. In particular embodiments, the modified HSCs or HSPCs are infused into the subject, e.g., by intravenous infusion, e.g., through a central vein over a period of several minutes to several hours. In particular embodiments, the modified HSCs or HSPCs transiently express the modified CD117 polypeptide, which is constitutively active, e.g., has constitutive kinase activity. In certain embodiments, the modified CD117 has constitutive autophosphorylation activity, e.g., without bound SCF. In particular embodiments, the modified HSCs or HSPCs that transiently express the modified CD117 are resistant to ablation by an anti-c-Kit antibody, such as, e.g., JSP191. Accordingly, a subject in need of HCT may be conditioned using an anti-c-Kit monoclonal antibody such as JSP191 prior to, with, or following HCT in order to ablate diseased HSPCs, whereas the transplanted modified HSCs or HSPCs are less susceptible to ablation by any monoclonal antibody in the subject following transplant, since they transiently express the modified CD117, which provides transient constitutive CD117 signaling, even in the presence of the antibody.

Where the donor is allogeneic to the recipient, the HLA type of the donor and recipient may be tested for a match, or haploidentical cells may be used. In certain embodiments, cells obtained from HLA-haploidentical donors or HLA-identical donors are used. HLA-haploidentical donors can be manipulated by CD34 or CD34/CD90 selection. For HLA matching, traditionally, the loci critical for matching are HLA-A, HLA-B, and HLA-DR. HLA-C and HLA-DQ are also now considered when determining the appropriateness of a donor. A completely matched sibling donor is generally considered the ideal donor. For unrelated donors, a complete match or a single mismatch is considered acceptable for most transplantation, although in certain circumstances, a greater mismatch is tolerated. Preferably matching is both serologic and molecular. Where the donor cells are from umbilical cord blood, the degree of tolerable HLA disparity is much greater, and a match of three or four out of the six HLA-A, HLA-B and HLA-DRB1 antigens is typically sufficient for transplantation. Immunocompetent donor T cells may be removed using a variety of methods to reduce or eliminate the possibility that graft versus host disease (GVHD) will develop.

The HCT methods disclosed use modified HSCs comprising a modified CD117 polypeptide or nucleic acid encoding the modified CD117 polypeptide. The methods are believed to result in reduced toxicity, reduced morbidity, or reduced graft-versus-host disease, as compared to HCT wherein a subject is administered HSCs that do not comprise the modified CD117 polypeptide or nucleic acid encoding the modified CD117 polypeptide. The methods of the invention are also believed to provide for improved engraftment of stem cells after transplantation into a recipient.

In particular embodiments of any of the methods of treatment disclosed herein, the subject is administered a conditioning regimen to facilitate or increase engraftment of the modified cells. In certain embodiments, the conditioning regimen depletes endogenous normal or disease HSCs of the subject. Conditioning regimens may be given prior to transplant to reduce the number of blood stem cells in the bone marrow to make space for donor blood stem cells to engraft and cure the patient. Typically, the conditioning regimen is administered prior to and/or concurrent with the administering of the pharmaceutical composition. In certain embodiments, the conditioning regimen comprises administration of an anti-CD117 antibody, wherein the anti-CD117 antibody depletes endogenous HSCs expressing wild-type CD117, but the anti-CD117 antibody does not deplete the administered modified HSCs. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. In particular embodiments, the conditioning regimen comprises an anti-CD117 antibody alone. In particular embodiments, the subject is administered the anti-CD117 antibody prior to administration of the modified HSCs, e.g., as a single dose.

An effective dose of anti-c-Kit antibody is the dose that depletes endogenous hematopoietic stem cells. The effective dose will depend on the individual and the specific antibody, but it will generally be up to about 100 μg/kg body weight, up to about 250 μg/kg, up to about 500 μg/kg, up to about 750 μg/kg, up to about 1 mg/kg, up to about 1.2 mg/kg, up to about 1.5 mg/kg, up to about 3 mg/kg, up to about 5 mg/kg, up to about 10 mg/kg. In some embodiments, the subject is administered about 0.01 mg/kg to about 2 mg/kg of the anti-c-kit antibody, e.g., JSP191, and optionally the subject is administered about 0.1 mg/kg to about 1 mg/kg of the anti-c-Kit antibody, e.g., JSP191. In some embodiments, anti-c-Kit antibody may be administered to a subject in a dose about 0.01 mg/kg to about 2 mg/kg of the subject's body weight, or about 0.1 mg/kg to about 1 mg/kg of the subject's body weight. In some embodiments, the anti-c-Kit signaling antibodies are administered in a dose of about 0.6 mg/kg.

In certain embodiments, the conditioning regimen comprises administration of an anti-CD117 antibody in combination with one or more additional antibodies. In certain embodiments, the one or more additional antibodies comprise one or more of: anti-CD47, anti-CD40L, anti-CD122, anti-CD4, and/or anti-CD8 antibody.

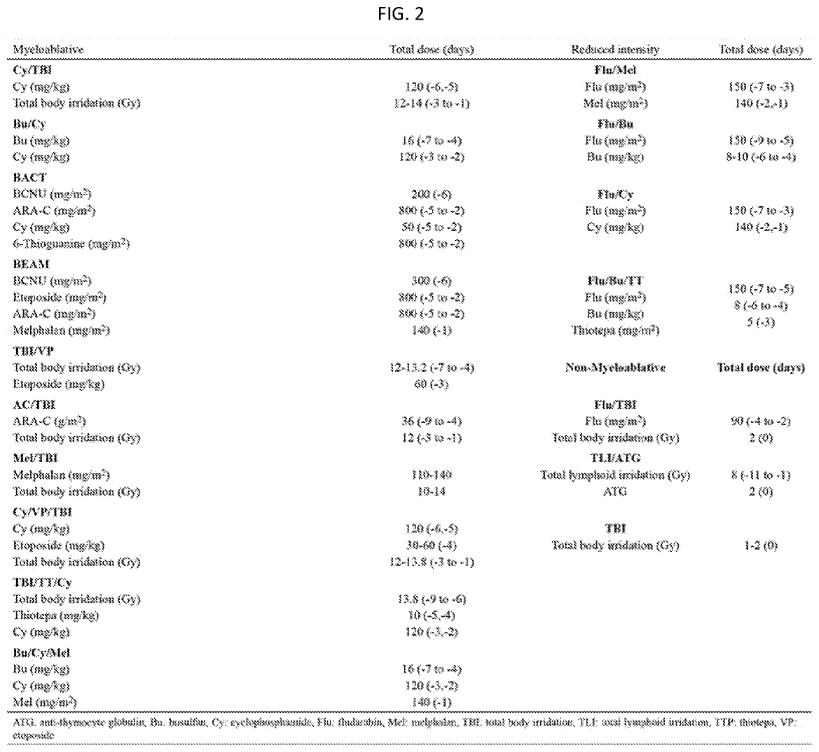

In certain embodiments, the conditioning regimen comprises administration of an anti-CD117 antibody, alone or in combination with a myeloablative (MA) conditioning, reduced intensity conditioning (RIC), or other non-MA (NMA) conditioning regimen. Examples of various conditioning regimens are provided in FIG. 2. In certain embodiments, the conditioning regimen is a genotoxic conditioning regimen and/or may comprise one or more of: chemotherapy (optionally a nucleoside analog and/or an alkylating agent), monoclonal antibody therapy, and radiation, optionally radiation to the entire body. In particular embodiments, since the subject is being administered modified cells, e.g., HSCs, comprising a modified CD117 described herein and/or an anti-CD117 antibody, the conditioning regimen is milder than would be used if the subject was being administered cells, e.g., HSCs, that did not comprise the modified CD117 polypeptide. In particular embodiments, wherein the conditioning regimen comprises use of an anti-CD117 antibody in combination with chemotherapy (optionally a nucleoside analog and/or an alkylating agent), other monoclonal antibody therapy, and/or radiation, the amount of chemotherapy, other monoclonal antibody therapy, and/or radiation is reduced as compared to the amount used when not in combination with an anti-CD117 antibody, such as JSP191. For example, either or both the amount and/or duration of other conditioning therapy may be reduced by at least or about 20%, at least or about 30%, at least or about 40%, at least or about 50%, at least or about 60%, at least or about 70%, at least or about 80%, at least or about 90%, or by about 100%.

However, in other embodiments, the subject is not administered a myeloablative or genotoxic conditioning regimen prior to or concurrent with the administering of the pharmaceutical composition. For example, the recipient may be immunocompetent, and the transplantation may be performed in the absence of myeloablative conditioning, i.e., in the absence of radiation and/or chemotherapeutic drugs. The recipient may be conditioned with the combined administration a set of agents selected according to the cells and HLA match.

The dose of stem cells, e.g., modified HSCs comprising a modified CD117 polypeptide and/or nucleic acid encoding a modified CD117 polypeptide, administered to a subject may depend on the purity of the infused cell composition, and the source of the cells. In particular embodiments, the dose administered is at least or about 1-2×10⁶ CD34+ cells/kg body weight for autologous and allogeneic transplants. Higher doses can include, for example, at least or about 3×10⁶, at least or about 4×10⁶, at least or about 5×10⁶, at least or about 6×10⁶, at least or about 7×10⁶, at least or about 8×10⁶, at least or about 9×10⁶, at least or about 10′ or more CD34+ cells/kg body weight for autologous and allogeneic transplants. Frequently, the dose is limited by the number of available cells, and the methods disclosed encompass delivering less cells when necessary or limited. Typically, regardless of the source, the dose is calculated by the number of CD34+ cells present. The percent number of CD34+ cells can be low for unfractionated bone marrow or mobilized peripheral blood; in which case the total number of cells administered may be higher.

In certain embodiments, a maximum number of CD3+ cells delivered with the modified HSC composition is not more than about 10⁷ CD3+ cells/kg of recipient body weight, not more than about 10⁶ CD3+ cells/kg of recipient body weight, not more than about 10⁵ CD3+ cells/kg of recipient body weight, or not more than about 10⁴ CD3+ cells/kg of recipient body weight. Alternatively, cell populations may be selected for expression of CD34 and CD90, which cell populations may be highly purified, e.g., at least about 85% CD34+ CD90+ cells, at least about 90% CD34+ CD90+ cells, at least about 95% CD34+ CD90+ cells and may be up to about 99% CD34+ CD90+ cells or more.

In certain embodiments, the disclosure includes a method of treating a mammalian subject in need thereof, comprising administering to the subject modified cells, e.g., HSCs and/or HSPCs, comprising a modified CD117 polypeptide disclosed herein, e.g., a modified CD117 with constitutive c-Kit signaling or kinase activity. In certain embodiments, the modified CD117 has constitutive autophosphorylation activity, e.g., without bound SCF. In particular embodiments, the modified CD117 polypeptide is transiently expressed in the cells, e.g., for about one day, about two days, about three days, about four days, about five days, or about a week. In particular embodiments, the subject is also administered a conditioning regimen to facilitate or increase engraftment of the modified cells following transplantation, wherein the conditioning regimen is administered prior to or concurrent with the administering of the pharmaceutical composition. In particular embodiments, the conditioning regimen comprises administration of an anti-c-Kit antibody, e.g., any disclosed herein (such as, e.g., JSP191), to the subject. In some embodiments, the anti-c-Kit antibody is administered to the subject prior to administration of the pharmaceutical composition to the subject. In particular embodiments, there is a “washout” period following administration of the anti-c-Kit antibody and before administration of the modified cells (i.e., the HCT). This period of time allows clearance of the anti-c-Kit antibody (or other agent used for conditioning). The period of time required for clearance of the ablative agent may be empirically determined, or may be based on prior experience of the pharmacokinetics of the agent. Historically, the time for clearance was usually the time sufficient for the level of ablative agent, e.g., anti-c-Kit antibody, to decrease at least about 10-fold from peak levels, usually at least about 100-fold, 1000-fold, 10,000-fold, or more. However, since the modified cells being administered to the subject according to the methods disclosed herein comprise a modified CD117 polypeptide that is not bound by the ablative anti-c-Kit antibody used for conditioning, the disclosed methods do not require a wash-out period, or they require only a reduced wash-out period as compared to when unmodified cells are transplanted. In certain embodiments, the wash-out period is less than five days, less than four days, less than 3 days, less than two days, or less than one day. In certain embodiments, the method comprises administering the anti-c-Kit antibody and the pharmaceutical composition or modified cells, e.g., modified HSCs and/or HSPCs, during an overlapping period of time or at about the same time. In particular embodiments, the method comprises administering the anti-c-Kit antibody to the subject after administration of the pharmaceutical composition or modified cells, e.g., modified HSCs and/or HSPCs, optionally for a period of time of at least one day, at least two days, at least three days, at least four days, at least five days, or at least one week. This may continue to ablate endogenous HSCs and/or HSPCs following administration of the modified HSCs and/or HSPCs, thus allowing greater engraftment.

In one embodiment, the method comprises:
(i) selectively ablating endogenous hematopoietic stem cells in the subject by administering to the subject an anti-c-Kit antibody, e.g., JSP-191;
(ii) optionally, waiting for a period of time following administration of the anti-c-Kit antibody; and
(iii) following (ii), administering to the subject the pharmaceutical composition comprising the modified cells, e.g., modified HSCs and/or modified HSPCs, in a dose effective to achieve multilineage peripheral blood chimerism.
   In particular embodiments, the period of time of step (ii) is less than five days, less than four days, less than 3 days, less than two days, or less than one day, or there is no period of time. In particular embodiments, the modified CD117 has constitutive c-Kit signaling or kinase activity. In particular embodiments, the modified CD117 polypeptide is transiently expressed in the cells, e.g., for about one day, about two days, about three days, about four days, about five days, or about a week. In particular embodiments, the modified CD117 polypeptide has at least 90%, at least 95%, at least 98%, or at least 99% identity to a wild-type human c-Kit polypeptide and comprises an amino acid substitution at a position referred to as N505 or D816, such as, e.g., N505I or D816V.

In certain embodiments, the method of treating a subject in need of HCT comprises:
(i) administering a conditioning regimen to the subject, wherein the conditioning regimen comprises an anti-CD117 monoclonal antibody, e.g., JSP191; and
(ii) administering modified HSCs to the subject, wherein the modified HSCs comprise a modified CD117 polypeptide, wherein the modified CD117 polypeptide is expressed on the cell surface, and wherein the modified HSCs are not depleted by the conditioning regimen to the same extent as endogenous HSCs that comprise only wild type CD117 polypeptide and/or wherein the modified HSCs have a proliferative advantage as compared to the endogenous HSCs. In particular embodiments, the modified CD117 has constitutive c-Kit signaling or kinase activity. In certain embodiments, the modified CD117 has constitutive autophosphorylation activity, e.g., without bound SCF. In particular embodiments, the modified CD117 polypeptide is transiently expressed in the cells, e.g., for about one day, about two days, about three days, about four days, about five days, or about a week. In particular embodiments, the conditioning regimen comprises a monoclonal anti-c-Kit antibody, e.g., JSP191. In particular embodiments, the modified CD117 polypeptide has at least 90%, at least 95%, at least 98%, or at least 99% identity to a wild-type human c-Kit polypeptide and comprises an amino acid substitution at a position referred to as N505 or D816, such as, e.g., N505I or D816V. The actual location of the “N505” or “D816” modification may differ depending on the particular isotype of c-Kit polypeptide. In particular embodiments, the modified CD117 polypeptide has the sequence shown in any one of SEQ ID NOs:3-6.

In some embodiments, the transplantation is performed in the absence of myeloablative conditioning. In some embodiments the recipient is immunocompetent. The administration of the pre-transplantation conditioning regimen is repeated as necessary to achieve the desired level of ablation. Following transplantation with donor stem cells, the recipient may be a chimera or mixed chimera for the donor cells.

The methods disclosed herein may be used to treat a variety of indications amenable to stem cell transplantation. In particular embodiments, HCT methods disclosed herein are used to treat a disease or disorder selected from the group consisting of: a cancer, a cardiac disorder, a neural disorder, an autoimmune disease, an immunodeficiency, a metabolic disorder, hemoglobinopathies, and a genetic disorder. In particular embodiments, they are used to treat any of the following disorders: multiple myeloma, non-Hodgkin lymphoma, Hodgkin disease, acute myeloid leukemia, neuroblastoma, germ cell tumors, and autoimmune disorders, e.g., systemic lupus erythematosus (SLE), systemic sclerosis, or amyloidosis, for example, by autologous HCT. In particular embodiments, they are used to treat any of the following disorders: acute myeloid leukemia, acute lymphoblastic leukemia, chronic myeloid leukemia; chronic lymphocytic leukemia, myeloproliferative disorders, myelodysplastic syndromes, multiple myeloma, non-Hodgkin lymphoma, Hodgkin disease, aplastic anemia, pure red cell aplasia, paroxysmal nocturnal hemoglobinuria, Fanconi anemia, thalassemias, thalassemia major, sickle cell anemia, combined immunodeficiency, severe combined immunodeficiency (SCID), Wiskott-Aldrich syndrome, hemophagocytic lymphohistiocytosis (HLH), inborn errors of metabolism (e.g., mucopolysaccharidosis, Gaucher disease, metachromatic leukodystrophies, and adrenoleukodystrophies), epidermolysis bullosa, severe congenital neutropenia, Shwachman-Diamond syndrome, Diamond-Blackfan anemia, leukocyte adhesion deficiency, and the like, for example, by allogeneic HCT.

In particular embodiments, the methods disclosed are used to treat a solid tissue cancer or a blood cancer, such as a leukemia, a lymphoma, or a myelodysplastic syndrome. In particular embodiments, the leukemia is acute myeloid leukemia (AML),

In particular embodiments, the lymphoma is diffuse large B-cell lymphoma.

In particular embodiments, the methods disclosed are used to treat an immunodeficiency. In particular embodiments, the immunodeficiency is severe combined immunodeficiency (SCAM. In particular embodiments, the immunodeficiency is immunoglobulin G subclass deficiency, selective immunoglobulin A deficiency, DiGeorge syndrome, hyper-immunoglobulin M (HIGM) syndrome, selective IgM deficiency, Wiskott-Aldrich syndrome, or X-linked agammaglobulinemia (XLA).

In particular embodiments, the methods disclosed are used to treat a genetic disorder. In particular embodiments, the genetic disorder is sickle cell disease or Fanconi anemia. Sickle cell diseases that may be treat include, but are not limited to: HbS disease; drepanocytic anemia; meniscocytosis, and chronic hemolytic anemia.

In certain embodiments of any of the HCT methods disclosed, the method further comprises administering to the subject a therapeutic agent for treatment of the disease or disorder being treated by the HCT method.

### EXAMPLES

### Example 1

### Proliferation of Hematopoietic Cells Expressing a CD117 Variant is not Inhibited by an Anti-CD117 Antibody

To demonstrate that expression of a mutated CD117 confers a proliferative advantage for hematopoietic cells expressing mutant CD117 vs. wild-type CD117, Ba/F3 cells expressing wild-type human CD117 (c-Kit) and mutant human CD117-D816V were cultured in the absence of IL-3, in varying concentrations of stem cell factor (SCF), and in the presence or absence of anti-CD117 antibody JSP191.

Control parental Ba/F3 cells did not proliferate in the absence of IL-3. Further, parental Ba/F3 cells did not express CD117 and were not responsive to SCF signaling. Therefore, control parental Ba/F3 cells did not proliferate in the presence of increasing concentrations of SCF, and there was no effect on viability or proliferation with the addition of JSP191.

Ba/F3 cell line expressing wild-type human CD117 (c-Kit) showed dose-responsive proliferation to SCF, which was inhibited in the presence of anti-CD117 antibody JSP191.

Ba/F3 cell line expressing the CD117-D816V mutant was able to proliferate in the absence of SCF and proliferation was not inhibited by the presence of the anti-CD117 antibody JSP191.

These studies demonstrate that cells comprising a constitutively active modified CD117 are capable of proliferating in the presence of anti-CD117 antibodies that inhibit SCF binding to CD117, and thus the modified CD117 confers a proliferative advantage to the modified cells as compared to wild type cells, particularly in the presence of the anti-CD117 antibody.

The various embodiments described above can be combined to provide further embodiments.

Aspects of the embodiments can be modified, if necessary to employ concepts of the various patents, patent applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

All of the U.S. patents, U.S. patent application publications, U.S. patent applications, foreign patents, foreign patent applications, and non-patent publications referred to in this specification and/or listed in the Application Data Sheet are incorporated herein by reference, in their entirety.

1. A pharmaceutical composition comprising a pharmaceutically acceptable excipient, carrier, or diluent and a modified hematopoietic stem cell (HSC) or a hematopoietic stern and progenitor cell (HSPC), wherein the modified HSC or HSPC comprises a modified CD117 polypeptide, optionally wherein the modified CD117 polypeptide has constitutive c-Kit signaling and/or kinase activity, and optionally wherein the modified cell is capable of proliferation and/or survival when contacted with an anti-c-Kit monoclonal antibody capable of inhibiting proliferation and/or survival of an HSPC expressing only a wild-type CD117.

2. The pharmaceutical composition of claim 1, wherein the c-Kit signaling and/or kinase activity of the modified CD117 is not substantially inhibited by an anti-c-Kit monoclonal antibody.

3. The pharmaceutical composition of claim 2, wherein the anti-c-Kit monoclonal antibody inhibits binding of SCF to CD117.

4. The pharmaceutical composition of claim 2, wherein the anti-c-Kit monoclonal antibody comprises one or more of the six CDRs present in any one of JSP191, AB85, CDX-0159, or FSI-174.

5. The pharmaceutical composition of claim 2, wherein the anti-c-Kit monoclonal antibody is any one of JSP191, AB85, CDX-0159, or FSI-174.

6. The pharmaceutical composition of claim 5, wherein the anti-c-Kit antibody is JSP191.

7. The pharmaceutical composition of claim 5, wherein the anti-c-Kit antibody is FSI-174.

8. The pharmaceutical composition of any one of claims 1-7, wherein the modified CD117 comprises one or more amino acid modifications as compared to the wild-type CD117 polypeptide.

9. The pharmaceutical composition of claim 8, wherein the one or more amino acid modifications comprise one or more amino acid substitutions, insertions, or deletions.

10. The pharmaceutical composition of claim 8, wherein one or more of the amino acid modifications are present within surface exposed amino acid residues of the extracellular domain, within the membrane spanning domain, or within an intracellular domain of the modified CD117 polypeptide.

11. The pharmaceutical composition of claim 8, wherein the modified CD117 polypeptide comprises substitution or deletion of one or more of the following amino acids present in wild type human CD117: N505 or D816.

12. The pharmaceutical composition of claim 11, wherein the modified CD117 polypeptide comprises a D816V substitution and/or a N505I substitution as compared to wild type human CD117.

13. The pharmaceutical composition of any one of claims 1-12, wherein the modified CD117 polypeptide has at least 90%, at least 95%, at least 98%, or at least 99% sequence homology to wild type CD117 polypeptide.

14. The pharmaceutical composition of any one of claims 8-13, wherein the wild type CD117 polypeptide is a wild type human CD117 polypeptide, optionally having one of the following amino acid sequences:
(SEQ ID NO: 1)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
or
(SEQ ID NO: 2)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNDSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV.

15. The pharmaceutical composition of any one of claims 1-14, wherein the modified cell expresses both the modified CD117 polypeptide and a wild type CD117 polypeptide.

16. The pharmaceutical composition of any one of claims 1-15, wherein the modified cell expresses the modified CD117 polypeptide transiently.

17. The pharmaceutical composition of any one of claims 1-16, wherein the HSC or HSPC is CD34+, optionally wherein the HSPC is CD34+/CD90+, CD34+/CD38−, or CD34+/CD38−/CD90+, or CD34+CD133+.

18. The pharmaceutical composition of any one of claims 1-17, wherein the cell is a human cell.

19. The pharmaceutical composition of any one of claims 1-18, wherein the cell was obtained from a mammalian donor.

20. The pharmaceutical composition of claim 19, wherein the mammalian donor is a subject in need of a hematopoietic cell transplant (HCT).

21. The pharmaceutical composition of claim 19, wherein the mammalian donor is a healthy donor.

22. The pharmaceutical composition of any one of claims 19-21, wherein the cell obtained from the mammalian donor was modified ex vivo.

23. The pharmaceutical composition of any one of claims 1-22, further comprising an anti-CD117 antibody.

24. The pharmaceutical composition of any one of claims 1-23, further comprising one or more anti-CD47, anti-CD40L, anti-CD122, anti-CD4, and/or anti-CD8 antibody.

25. A method of treating a mammalian subject in need thereof, comprising administering to the subject the pharmaceutical composition of any one of claims 1-24.

26. The method of claim 25, wherein the subject is administered a conditioning regimen to facilitate or increase engraftment of the modified cells, wherein the conditioning regimen is administered prior to or concurrent with the administering of the pharmaceutical composition.

27. The method of claim 26, wherein the conditioning regimen is also administered subsequent to the administering of the pharmaceutical composition.

28. The method of claim 26 or claim 27, wherein the conditioning regimen is milder than would be used if the subject was being administered hematopoietic stern cells that did not comprise the modified CD117 polypeptide.

29. The method of claim 25, wherein the subject is not administered a conditioning regimen to facilitate or increase engraftment of the modified cells, prior to or concurrent with the administering of the pharmaceutical composition.

30. The method of any one of claims 25-29, wherein the method results in reduced toxicity, reduced morbidity, or reduced graft-versus-host disease, as compared to a method wherein a subject is administered hematopoietic stem cells that do not comprise the modified CD117 polypeptide in combination with a conditioning regimen.

31. The method of any one of claims 25-30, wherein the conditioning regimen comprises or consists of administration of an anti-CD117 monoclonal antibody, optionally JSP191.

32. The method of any one of claims 25-31, wherein the subject is treated for a disease or disorder selected from the group consisting of: a cancer, a cardiac disorder, a neural disorder, an autoimmune disease, an immunodeficiency, a metabolic disorder, and a genetic disorder.

33. The method of claim 32, wherein the cancer is a solid tissue cancer or a blood cancer.

34. The method of claim 33 wherein the blood cancer is a leukemia, a lymphoma, or a myelodysplastic syndrome.

35. The method of claim 34, wherein the leukemia is acute myeloid leukemia (AML).

36. The method of claim 32, wherein the immunodeficiency is severe combined immunodeficiency (SCID).

37. The method of claim 32, wherein the genetic disorder is sickle cell disease or Fanconi anemia.

38. The method of any one of claims 25-37, further comprising administering to the subject a therapeutic agent for treatment of the disease or disorder.

39. A modified CD117 polypeptide comprising one or more amino acid modifications as compared to a wild type CD117 polypeptide, wherein an anti-c-Kit monoclonal antibody does not substantially inhibit or reduce c-Kit signaling, optionally in response to SCF binding, by the modified CD117 polypeptide expressed in cells as compared to the wild type CD117 polypeptide.

40. The modified CD117 polypeptide of claim 39, wherein the modified CD117 polypeptide substantially retains kinase activity, optionally in response to SCF binding, as compared to the wild type CD117 polypeptide.

41. The modified CD117 polypeptide of claim 39 or claim 40, wherein the modified CD117 polypeptide has constitutive c-Kit signaling and/or kinase activity.

42. The modified CD117 polypeptide of any one of claims 39-41, wherein the modified CD117 polypeptide substantially has increased c-Kit signaling and/or kinase activity, optionally in response to SCF binding, as compared to the wild type CD117 polypeptide.

43. The modified CD117 polypeptide of any one of claims 39-42, wherein the anti-c-Kit antibody comprises one or more of the six CDRs present in any one of JSP191, AB85, CDX-0159, or FSI-174.

44. The modified CD117 polypeptide of claim 43, wherein the anti-c-Kit antibody is any one of JSP191, AB85, CDX-0159, or FSI-174.

45. The modified CD117 polypeptide of claim 43, wherein the anti-c-Kit antibody comprises one or more, optionally six, CDRs present in JSP191 and/or FSI-174.

46. The modified CD117 polypeptide of claim 45, wherein the anti-c-Kit antibody is JSP191 or FSI-174.

47. The modified CD117 polypeptide of any one of claims 39-46, wherein the one or more amino acid modifications comprise one or more amino acid substitutions, insertions, or deletions.

48. The modified CD117 polypeptide of claim 47, wherein one or more of the amino acid modifications are present within surface exposed amino acid residues of the extracellular domain, within the membrane spanning domain, or within an intracellular domain of the wild type CD117 polypeptide.

49. The modified CD117 polypeptide of claim 47 or claim 48, wherein the one or more amino acid modifications comprise one or more amino acid substitutions or deletions.

50. The modified CD117 polypeptide of claim 49, wherein the one or more amino acid modifications comprise one or more amino acid substitutions.

51. The modified CD117 polypeptide of any one of claims 47-50, wherein the one of more amino acid substitutions or deletions comprises substitution or deletion of one or more of the following amino acids present in wild type human CD117: N505 or D816.

52. The modified CD117 polypeptide of claim 50 or claim 51, wherein the one of more amino acid substitutions comprises a D816V substitution and/or a N505I substitution.

53. The modified CD117 polypeptide of any one of claims 39-52, wherein the modified CD117 polypeptide has at least 90%, at least 95%, at least 98%, or at least 99% sequence homology to the wild type CD117 polypeptide.

54. The modified CD117 polypeptide of any one of claims 39-53, wherein the wild type CD117 polypeptide is a wild type human CD117 polypeptide, optionally having one of the following amino acid sequences:
(SEQ ID NO: 1)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKGNNKEQIHPHTLFTPLLIGFVIVAGMMCIIVMILTYKYLQK
PMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEA
ALYKNLLHSKESSCSDSTNEYMDMKPGVSYVVPTKADKRRSVRIGSYIER
DVTPAIMEDDELALDLEDLLSFSYQVAKGMAFLASKNCIHRDLAARNILL
THGRITKICDFGLARDIKNDSNYVVKGNARLPVKWMAPESIFNCVYTFES
DVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPV
VDHSVRINSVGSTASSSQPLLVHDDV;
or
(SEQ ID NO: 2)
MRGARGAWDFLCVLLLLLRVQTGSSQPSVSPGEPSPPSIHPGKSDLIVRV
GDEIRLLCTDPGFVKWTFEILDETNENKQNEWITEKAEATNTGKYTCTNK
HGLSNSIYVFVRDPAKLFLVDRSLYGKEDNDTLVRCPLTDPEVTNYSLKG
CQGKPLPKDLRFIPDPKAGIMIKSVKRAYHRLCLHCSVDQEGKSVLSEKF
ILKVRPAFKAVPVVSVSKASYLLREGEEFTVTCTIKDVSSSVYSTWKREN
SQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIY
MNRTFTDKWEDYPKSENESNIRYVSELHLTRLKGTEGGTYTFLVSNSDVN
AAIAFNVYVNTKPEILTYDRLVNGMLQCVAAGFPEPTIDWYFCPGTEQRC
SASVLPVDVQTLNSSGPPFGKLVVQSSIDSSAFKHNGTVECKAYNDVGKT
SAYFNFAFKEQIHPHTLFTPLLIGFVIVAGMMCIIVMILTYKYLQKPMYE
VQWKVVEEINGNNYVYIDPTQLPYDHKWEFPRNRLSFGKTLGAGAFGKVV
EATAYGLIKSDAAMTVAVKMLKPSAHLTEREALMSELKVLSYLGNHMNIV
NLLGACTIGGPTLVITEYCCYGDLLNFLRRKRDSFICSKQEDHAEAALYK
NLLHSKESSCSDSTNEYMDMKPGVSYVVPTKADKRRSVRIGSYIERDVTP
AIMEDDELALDLEDLLSFSYQVAKGMAFLASKNCIHRDLAARNILLTHGR
ITKICDFGLARDIKNDSNYVVKGNARLPVKWMAPESIFNCVYTFESDVWS
YGIFLWELFSLGSSPYPGMPVDSKFYKMIKEGFRMLSPEHAPAEMYDIMK
TCWDADPLKRPTFKQIVQLIEKQISESTNHIYSNLANCSPNRQKPVVDHS
VRINSVGSTASSSQPLLVHDDV.

55. A nucleic acid encoding the modified CD117 polypeptide of any one of claims 39-54.

56. The nucleic acid of claim 55, wherein the nucleic acid comprises RNA, DNA, or a combination thereof.

57. The nucleic acid of claim 56, wherein the nucleic acid comprises a modified mRNA.

58. The nucleic acid of any one of claims 55-57, wherein the nucleic acid is associated with one or more lipids, optionally wherein the nucleic acid is present within a lipid nucleic acid particle, a lipid nanoparticle, or a liposome.

59. A vector comprising the nucleic acid of any one of claims 55-58.

60. The vector of claim 59, wherein the vector is an expression vector.

61. The vector of claim 59 or claim 60, wherein the vector is a viral vector, optionally an AAV vector or a lentiviral vector.

62. The vector of any one of claims 59-61, wherein the vector is capable of transducing hematopoietic stem cells.

63. A modified cell comprising the modified CD117 polypeptide of any one of claims 39-54 and/or the nucleic acid of any one of claims 55-58.

64. The modified cell of claim 63, wherein the cell expresses both the modified CD117 polypeptide and a wild type CD117 polypeptide.

65. The modified cell of claim 63 or claim 64 wherein the cell was transduced with the vector of any one of claims 59-62.

66. The modified cell of any one of claims 63-65, wherein the cell is a stem cell or a pluripotent cell.

67. The modified cell of claim 66, wherein the stem cell is a hematopoietic stem cell (HSC) or a hematopoietic stern and progenitor cell (FISPC).

68. The modified cell of any one of claims 63-67, wherein the cell is CD34+, optionally wherein the cell is CD34+/CD90+, CD34+/CD38−, or CD34+/CD38−/CD90+, or CD34+CD133+.

69. The modified cell of any one of claims 63-68, wherein the cell is a human cell.

70. The modified cell of any one of claims 63-69, wherein the cell was obtained from a mammalian donor.

71. The modified cell of claim 70, wherein the mammalian donor is a subject is in need of a hematopoietic cell transplant (HCT).

72. The modified cell of claim 70, wherein the mammalian donor is a healthy donor.

73. The modified cell of any one of claims 70-72, wherein the cell obtained from the mammalian donor was modified ex vivo.

74. The modified cell of any one of claims 63-73, wherein the cell expresses the modified CD117 polypeptide, optionally wherein the modified cell expresses the modified CD117 polypeptide transiently.

75. The modified cell of claim 74, wherein the modified CD117 polypeptide is expressed on the cell surface or in the cell membrane.

76. The modified cell of any one of claims 63-75, wherein the cell is capable of proliferating and/or surviving in the presence of an anti-CD117 antibody.

77. The modified cell of claim 76, wherein the anti-CD117 antibody is capable of inhibiting proliferation and/or survival of a cell expressing only the wild-type CD117.

78. The modified cell of claim 76 or claim 77, wherein the anti-CD117 antibody is selected from the group consisting of: JSP191, CDX-0159, AB85, and FSI-174.

79. A method of modifying a cell, comprising introducing the nucleic acid of any one of claims 55-58 or the vector of any one of claims 59-62 into the cell, optionally wherein the cell is transiently modified, and optionally wherein the method is for preparing modified cells for hematopoietic cell transplantation (HCT) into a mammalian subject.

80. The method of claim 79, wherein the nucleic acid or vector is introduced into the cell by transfection, transduction, infection, electroporation, or nanopore technology.
